(19) United States
(12) Patent Application Publication
(10) Pub. No.: US 2025/0381227 A1
(43) Pub. Date: Dec. 18, 2025

(54) AGGLOMERATES OF SOLUBLE WHEY PROTEIN AGGREGATES AND MEDICAL USES THEREOF

(71) Applicant: Arla Foods Amba, Viby J (DK)

(72) Inventors: Søren Bang NIELSEN, Viby J (DK); Marie OSTENFELD, Viby J (DK); Ulla Ramer MIKKELSEN, Viby J (DK); Gerrit VAN HALL, Kobenhavn N (DK); Camilla Bertel ANDERSEN, Viby J (DK); Tanja Christine JÆGER, Viby J (DK)

(21) Appl. No.: 18/875,427

(22) PCT Filed: Jun. 20, 2023
(86) PCT No.: PCT/EP2023/066652

§ 371 Date: Dec. 16, 2024

(30) Foreign Application Priority Data
Jun. 20, 2022 (EP) 22179878.8

(51) Int. Cl. A61K 35/20; A61K 9/00; A61K 9/19; A61P 3/00

(52) U.S. Cl. CPC A61K 35/20; A61K 9/0095; A61K 9/19; A61P 3/00

(57) ABSTRACT

The present invention relates to agglomerates of soluble whey protein aggregates for use in the treatment and/or prevention of one or more conditions linked to GLP-1 blood levels in a human subject.

### FIELD OF THE INVENTION

The present invention relates to agglomerates of soluble whey protein aggregates for use in the treatment and/or prevention of one or more conditions linked to GLP-1 blood levels in a human subject.

### BACKGROUND OF THE INVENTION

GLP-1 is known to affect a large number of biological processes in the human body.

The prevalence of overweight and obesity has increased significantly worldwide over the last decades, conditions that have huge individual as well as societal costs, both in regard to economy and health. Obesity develops when energy intake is greater than energy expenditure, the excess energy being stored mainly as fat in adipose tissue. During development of obesity, lipids also accumulate in non-adipose tissue, leading to further metabolic deterioration, like inflammation, reduced insulin sensitivity, etc.

Body weight loss and prevention of weight gain can be achieved by reducing energy intake or bioavailability, increasing energy expenditure and/or reducing storage as fat. There is a persisting need in the food industry to find better nutritional solutions to combat overweight and obesity.

Intestinal GLP-1 is an endogenous satiation signal; GLP-1 is secreted in response to food ingestion; intestinal GLP-1 secretion occurs within the time frame of a meal; peripheral injections of GLP-1 at physiological doses reduce meal size; antagonizing endogenous GLP-1 increases meal size (Krieger, 2020).

It is very plausible that intestinal GLP-1 reduces food intake via GLP-1R (R=receptor) present on vagal afferents (Krieger, 2020). The low circulating concentrations of GLP-1 after a meal do not, however, automatically exclude the possibility of endocrine effects of intestinal GLP-1. This is supported by the findings that GLP-1 primarily induces its incretin effect in an endocrine manner via GLP-1R activation in the pancreas. Relevant to GLP-1 effects on eating, GLP-1 may be in sufficient concentrations to activate GLP-1R in brain areas located outside the blood-brain barrier. Findings further indicate that endogenous GLP-1 of intestinal origin induces satiation via vagal, rather than central, GLP-1R (Krieger, 2020).

Treatment with GLP-1RAs (RA=receptor agonist) promote weight loss by reducing appetite/food craving and enhancing satiety, delaying gastric emptying, and by mechanisms modulating gut-to-brain communication in (Patel Chavez et al., 2022).

Global prevalence of type-2 diabetes has similarly increased significantly, both in adults and children. Some of the increase may be due to lifestyle factors paralleling the rising obesity rates due to alterations in dietary patterns. There is a persisting need in the food industry to further improve the nutritional solutions provided to diabetic subjects or subjects at risk for developing type-2 diabetes. Especially improving the insulin and glucagon profile in a diabetic or pre-diabetic subject.

It is well known that incretins, glucagon-like peptide-1 (GLP-1) and glucose-dependent insulinotropic polypeptide (GIP) have pleiotropic effects on a variety of tissues including pancreatic beta-cells, liver, artery, heart, neuron and adipose tissue. While there are various GLP-1 target tissues, GLP-1 plays a crucial role in pancreatic beta-cells; GLP-1 binds to the GLP-1 receptor (GLP-1R) in the beta-cell membrane and thereby enhances insulin secretion, suppresses apoptotic cell death and in-crease proliferation of beta-cells. Furthermore, GLP-1 suppresses glucagon secretion (from pancreatic alpha cell). Together, this leads to improved glycemic control (Kaneto et al., 2021).

In response to the ingestion of food, GLP-1 and GIP are secreted from the gastrointestinal tract and stimulate insulin secretion from pancreatic beta-cells. Both incretin hormones bind to each receptor in the beta-cell membrane, which leads to enhancing insulin secretion, reducing beta-cell apoptosis and promoting beta-cell proliferation. Such an action of incretin hormones, however, is significantly reduced under diabetic conditions in humans and rodents such as mice and rats. It has been reported that expression levels of incretin receptors are reduced under diabetic conditions, which is probably involved in the impaired incretin effects and the development of beta-cell failure found in T2DM (Kaneto et al., 2021).

Insulin secretion is regulated by various intracellular signals in beta-cells. Kaneto et al., 2021 report that circulating physiological concentration of GLP-1 directly stimulates insulin secretion from pancreatic beta-cells. GLP-1 stimulates insulin secretion in a PKC-dependent or PKA-dependent manner, depending on its concentration.

Apart from its actions on body weight and glucose, GLP-1 can also regulate blood cholesterol and triglycerides by numerous ways. Acute and long term treatment with either GLP-1 or its stable analogs reduce fasting as well as postprandial lipids in healthy as well as T2DM patients (Patel et al., 2014).

Another increasingly common global health issue is the occurrence of non-alcoholic fatty liver disease, by build-up of fat in the liver, leading to chronic liver disease. There is a persisting need in the food industry to find better nutritional solutions to combat the occurrence of this condition.

In a recent RCT including patients with NAFLD, GLP-1 RA semaglutide significantly reduced liver steatosis compared with placebo which, together with improvements in liver enzymes and metabolic parameters, suggests a positive impact on disease activity and metabolic profile. Their results showing that semaglutide reduces fat content in hepatocytes suggest the potential to reverse steatosis and halt fibrosis progression. (Flint et al., 2021). Taken together, it is likely that in the near future GLP-1RA will play a greater role among clinicians to treat patients with NAFLD (Patel Chavez et al., 2022).

Yet another health related global issue is the occurrence of cardiovascular events, which is one of the leading causes of death globally. Heart attacks and strokes are usually acute events and are mainly caused by a blockage that prevents blood from flowing to the heart or brain. There is a persisting need in the food industry to find better nutritional solutions to combat the occurrence of cardiovascular events.

GLP-1 receptors are expressed in endothelial and smooth muscle cells. In endothelial cells, incretin signaling improves the vascular relaxation response through eNOS expression and activity and retards the development of arteriosclerosis. Activation of GLP-1 signaling in arteries leads to the mitigation of inflammatory cytokines. In arterial cells, GLP-1 signaling improves the wall disorder induced by various factors including hyperglycemia and inflammatory cytokines. From a clinical perspective, a series of large-scale clinical trials have shown that GLP-1RAs have favorable effects against the onset of cardiovascular events (Kaneto et al., 2021; Zhao et al., 2015).

Yet another health related global issue is the loss of muscle mass and muscle strength, which considerably decreases the quality of life of a patient suffering from such a condition, such as patients suffering from muscle atrophy due to sarcopenia or cachexia. Furthermore, muscle atrophy deteriorates metabolic conditions as healthy muscle mass is beneficial to metabolic regulation. There is a persisting need in the food industry to find better nutritional solutions for patients suffering from such loss of muscle mass or muscle strength. In particular for subjects that are unable to perform exercise regimens, a nutritional solution would be needed that is effective on its own. This could also be effective in improving the anabolic response to nutrient intake. In addition, dietary supplements to optimize muscle responses to exercise/muscle contractions, are of high need.

GLP-1 RA have in rodent studies been shown to ameliorate muscle atrophy (Gurjar et al., 2020; Hong et al., 2019; Nguyen et al., 2020). GLP-1 infusions have further been demonstrated to overcome anabolic resistance to feeding in older human muscle, via enhanced postprandial microvascular perfusion (Abdulla et al., 2020). Therefore, enhanced muscle microvascular perfusion may be a mediator of the ameliorating effects of GLP-1 on muscle atrophy.

In regard to muscle microvascular perfusion, GLP-1 may increase cardiac and skeletal muscle microvascular blood volume, improve vascular function of the conduit vessels, and enhance insulin's metabolic action (Love et al., 2020; Subaran et al., 2014).

Muscle microvascular surface area determines substrate and hormonal exchanges between plasma and muscle interstitium. GLP-1 regulates glucose-dependent insulin secretion and has numerous extrapancreatic effects, including a salutary vascular action (Subaran et al., 2014). In a human trial it was concluded that acute GLP-1 infusion significantly recruits skeletal and cardiac muscle microvasculature in addition to relaxing the conduit artery in healthy humans. This could contribute to increased tissue oxygen, nutrient and insulin delivery and exchange and therefore better prandial glycaemic control and tissue function in humans (Subaran et al., 2014)

The object of the present invention is to improve the state of the art and to provide a nutritional solution that addresses the needs expressed above. As disclosed herein, this is facilitated by increasing the concentration GLP-1 in a human subject in need thereof.

### SUMMARY OF THE INVENTION

In a first aspect, the present invention provides agglomerates of soluble whey protein aggregates (AWA) or a dosage form containing an effective amount of AWA for use as a medicament.

In a further aspect, the present invention provides AWA or the dosage form containing an effective amount of AWA according for use in treating and/or preventing one or more disorders linked to GLP-1 blood levels in a human subject.

In a further aspect, the present invention provides AWA or the dosage form containing an effective amount of AWA, for use in treatment and/or prevention of one or more disorders linked to GLP-1 blood levels, wherein said AWA facilitates to one or more of
i. increased sensation of satiety and/or decreasing hunger
ii. reduced body weight
iii. improved glycemic control
iv. reduced glucagon secretion
v. enhanced insulin secretion
vi. reduced body and/or liver fat
vii. ameliorated muscle atrophy
viii. reduced risk of arteriosclerosis, hypertension, dyslipidemia, and/or cardiovascular events
in the human subject.

In a further aspect, the present invention provides AWA or the dosage form containing an effective amount of AWA for use in treatment and/or prevention of a disorders selected from one or more of:
a. type II diabetes,
b. pre-diabetes
c. hyperglycemia
d. Nonalcoholic fatty liver disease (NAFLD)
e. Nonalcoholic steatohepatitis (NASH)
f. overweight
g. obesity,
h. obesity related disorders,
i. muscle atrophy
j. cardio vascular disease
k. arteriosclerosis
l. hypertension
m. dyslipidemia

A second aspect of the invention provides a dosage form containing an effective amount of AWA. The dosage form may be in the form of a nutritional powder comprising AWA in an amount of at least 30% w/w relative to total protein, preferably wherein the nutritional powder furthermore comprises at least one non-dairy components, e.g. in the form of a non-dairy protein or other components that are not found milk or whey.

A third aspect of the invention provides a method of producing a composition comprising AWA, comprising the steps of
a. providing a whey protein solution:
   having a pH in the range of the 6-9,
   having a weight ratio between total protein and the total content of calcium and magnesium of at least 100
   comprising at least 1-10% w/w BLG relative to the weight of the whey protein solution
   comprising at least 30% BLG relative to total protein
b. subjecting the whey protein solution to heat-treatment which involves heating it to a temperature in the range of 68-180 degrees C. for a duration sufficient to denature at least 25% w/w of the BLG to obtain a suspension containing sWPA, and
c. subjecting the suspension or a protein concentrate thereof to processing that leads to agglomeration of the sWPA, preferably freeze-drying and preferably followed by particle size reduction of the freeze-dried product, thereby obtaining a composition comprising agglomerates of soluble whey protein aggregates.

A fourth aspect of the invention provides AWA or the dosage form containing an effective amount of AWA for use in treating and/or preventing one or more disorders linked to GLP-1 blood levels in a human subject, or the nutritional powder comprising AWA, wherein the AWA is obtainable by, and e.g. obtained by,
a. providing a whey protein solution:
   having a pH in the range of the 6-9,
   having a weight ratio between total protein and the total content of calcium and magnesium of at least 100
   comprising at least 1-10% w/w BLG relative to the weight of the whey protein solution
   comprising at least 30% BLG relative to total protein
b. subjecting the whey protein solution to heat-treatment which involves heating it to a temperature in the range of 68-180 degrees C. for a duration sufficient to denature at least 25% w/w of the BLG to obtain a suspension containing sWPA, and
c. subjecting the suspension or a protein concentrate thereof to processing that leads to agglomeration of the sWPA, thereby obtaining agglomerates of soluble whey protein aggregates.

Further aspects of the invention are apparent from the description of the invention.

### DESCRIPTION OF THE INVENTION

### Brief Description of the Figures

FIG. 1A: Incretin GLP-1 concentration in the post-absorptive state and 6 h following bolus ingestion of four protein ingredients: MCI, SPI, WPH and AWA by young, healthy individuals. MCI=Micellar casein isolate; SPI=Serum protein isolate (whey part from milk fractionation); WPH=hydrolyzed SPI; AWA=Agglomerates of soluble whey protein aggregates. Statistical differences (P<0.05) between ingredients within the young group are: AWA is significantly different from ▪ MCI, and ▴ WPH, and a strong tendency towards difference relative to ● SPI.

FIG. 1B: Incretin GLP-1 concentration in the post-absorptive state and 6 h following bolus ingestion of four protein ingredients: MCI, SPI, WPH and AWA by old, healthy individuals. MCI=Micellar casein isolate; SPI=Serum protein isolate (whey part from milk fractionation); WPH=hydrolyzed SPI; AWA=Agglomerates of soluble whey protein aggregates. Statistical differences (P<0.05) between ingredients within the old group are: AWA is significantly different from ▪ MCI, ● SPI, and ▴ WPH.

FIG. 2: Protein of total protein (%) in supernatant after centrifugation of sample at 3000×g for 5 min at pH 7.1, 4.2 and 2.9 for sWPA, AWA-f and AWA-C5b.

### ABBREVIATIONS, TERMS, AND DEFINITIONS

In the context of the present invention the term “soluble whey protein aggregates” (sWPA) pertains sub-micron sized aggregates of denatured whey proteins, which typically have linear, worm-like, branched or chain-like shapes. Soluble whey protein aggregate are capable of forming strong gels when present in sufficient concentration (much stronger than native whey protein or microparticulated whey protein) during acidification to pH 4.6. Soluble whey protein aggregates are well-known to the skilled person and are e.g. described in WO2007/110421A2 where they are referred to as linear aggregates, in WO06034856 wherein they are referred to as activated globular protein preparations, in WO2018/011392A1 where they are referred to as acid-gellable whey protein aggregates, in WO2021136785A1, or in US2008305235A1. The amount of soluble whey protein aggregate is quantified according to Analysis 3 of WO 2021/136785 A1. Aqueous solutions of soluble whey protein aggregates are typically transparent and soluble whey protein aggregates are therefore very well-suited for transparent beverages.

In the context of the present invention the term “agglomerates of soluble whey protein aggregates” or “AWA” pertains to sWPA which have been processed to provoke secondary aggregation between the sWPA and thereby agglomeration of sWPA. The inventors have found that the aggregation preferably is provoked by processing that brings the sWPA closer to each other to promote their interaction, e.g. by removing solvent (e.g. by freeze drying or concentration of solution of sWPA), and/or by reducing the electrostatic repulsion between the sWPA.

The formation of the AWA preferably involves one or more of:
Freeze-drying a suspension containing sWPA,
incubating a suspension contain sWPA under high ionic strength, e.g. by increasing the levels of divalent metal cations and/or increased levels of monovalent metal cations of a solution of sWPA having a low ionic strength,
Lowering the pH of a solution containing sWPA
Concentrating a solution containing sWPA, preferably by one or more of reverse osmosis, nanofiltration, ultrafiltration, microfiltration, evaporation, and a combination thereof.

Examples of production of AWA are e.g. found in U.S. Pat. No. 5,217,741 which is incorporated wherein by reference for all purposes.

In the context of the present invention, the term “beta-lactoglobulin” or “BLG” pertains to beta-lactoglobulin of mammal species, i.e. in native, unfolded and optionally also glycosylated forms and includes the naturally occurring genetic variants. The term also covers BLG prepared by fermentation. The term excludes denatured BLG. The content of BLG is measured according to Analysis 6 of WO 2021/136785 A1.

In the context of the present invention, the term “alpha-lactalbumin” or “ALA” pertains to alpha-lactalbumin of mammal species, i.e. in native, unfolded and optionally also glycosylated forms and includes the naturally occurring genetic variants. The term also covers ALA prepared by fermentation. The term excludes denatured, aggregated ALA. The content of ALA is measured according to Analysis 6 of WO 2021/136785 A1.

In the context of the present invention the term “Type 2 diabetes” or “T2D”, formerly known as adult-onset diabetes, is a form of diabetes that is characterized by high blood sugar, insulin resistance, and relative lack of insulin. Long-term complications from high blood sugar include heart disease, strokes, diabetic retinopathy which can result in blindness, kidney failure, and poor blood flow in the limbs which may lead to amputations. Type 2 diabetes primarily occurs as a result of obesity and lack of exercise. Two main characteristics of T2DM are the pancreatic beta-cell dysfunction and insulin resistance in various insulin target tissues such as the liver, skeletal muscle and adipose tissues. In a healthy, non-diabetic subject, beta-cells can compensate for insulin resistance by increasing insulin secretion or beta-cell mass, but insufficient compensation leads to the onset of T2DM. After then, once hyperglycemia becomes apparent, the beta-cell function gradually deteriorates and insulin resistance aggravates.

In the context of the present invention the term “prediabetes” is a component of the metabolic syndrome and is characterized by elevated blood sugar levels that fall below the threshold to diagnose diabetes mellitus. It usually does not cause symptoms but people with prediabetes often have obesity (especially abdominal or visceral obesity), dyslipidemia with high triglycerides and/or low HDL cholesterol, and hypertension. It is also associated with increased risk for cardiovascular disease (CVD). Prediabetes is more accurately considered an early stage of diabetes as health complications associated with type 2 diabetes often occur before the diagnosis of diabetes.

In the context of the present invention the term “hyperglycemia” is the medical term for a high blood sugar (glucose) level. It's a common problem for people with diabetes.

It can affect people with type 1 diabetes and type 2 diabetes, as well as pregnant women with gestational diabetes. It can occasionally affect people who do not have diabetes, but usually only people who are seriously ill, such as those who have recently had a stroke or heart attack, or have a severe infection. Hyperglycaemia should not be confused with hypoglycaemia, which is when a person's blood sugar level drops too low.

In the context of the present invention the term “non-alcoholic fatty liver disease” or “NAFLD” defines a range of conditions caused by a build-up of fat in the liver. It is usually seen in people who are overweight or obese. Early-stage NAFLD does not usually cause any harm, but it can lead to serious liver damage, including cirrhosis, if it gets worse. Having high levels of fat in your liver is also associated with an increased risk of serious health problems, such as diabetes, high blood pressure and kidney disease. If you already have diabetes, NAFLD increases your risk of developing heart problems. NAFLD is a hepatic manifestation of the metabolic syndrome and is characterized by excessive hepatic fat accumulation associated with insulin resistance.

In the context of the present invention the term “nonalcoholic steatohepatitis” or “NASH” is liver inflammation and damage caused by a buildup of fat in the liver. It is part of a group of conditions called nonalcoholic fatty liver disease. Many people have a buildup of fat in the liver, and for most people it causes no symptoms and no problems. But in some people, the fat causes inflammation and damages cells in the liver. Because of the damage, the liver doesn't work as well as it should. NASH can get worse and cause scarring of the liver, which leads to cirrhosis.

In the context of the present invention the term “obesity” is defined as a condition in which excess body fat has accumulated to such an extent that it may have a negative effect on health. People are classified as obese when their body mass index (BMI), a measurement obtained by dividing a person's weight by the square of the person's height is over 30 kg/m2; while the range 25-30 kg/m2 is defined as Overweight.

Obesity is a major cause of disability and is correlated with various diseases and conditions. In the context of the present invention the term “obesity related disorders” refer to such correlated diseases and disorders, such as cardiovascular diseases, type 2 diabetes, obstructive sleep apnea, certain types of cancer, and osteoarthritis.

In the context of the present invention the term “muscle atrophy” refers to the loss of skeletal muscle mass. It can be caused by immobility, aging, malnutrition, medications, or a wide range of injuries or diseases that impact the musculoskeletal or nervous system. Muscle atrophy also covers the terms sarcopenia and cachexia. Muscle atrophy results from an imbalance between protein synthesis and protein degradation. Muscle atrophy leads to muscle weakness and causes disability.

In the context of the present invention the term “cardiovascular disease” or “CVD” is used as a general term for conditions affecting the heart or blood vessels. This is usually associated with a build-up of fatty deposits inside the arteries (atherosclerosis) and an increased risk of blood clots. It can also be associated with damage to arteries in organs such as the brain, heart, kidneys and eyes.

In the context of the present invention the term “arteriosclerosis” is used in regard to the condition which occurs when the blood vessels that carry oxygen and nutrients from the heart to the rest of the body (arteries) become thick and stiff-sometimes restricting blood flow to organs and tissues. Healthy arteries are flexible and elastic, but over time, the walls in the arteries can harden, a condition commonly called hardening of the arteries. Atherosclerosis is a specific type of arteriosclerosis. Atherosclerosis is the buildup of fats, cholesterol and other substances in and on the artery walls. This buildup is called plaque. The plaque can cause the arteries to narrow, blocking blood flow. The plaque can also burst, leading to a blood clot.

In the context of the present invention the term “hypertension” refers to high blood pressure. If untreated, it increases risk of serious problems such as heart attacks and strokes. Blood pressure is recorded with two numbers. The systolic blood pressure (higher number) is the force at which the heart pumps blood around your body. The diastolic blood pressure (lower number) is the resistance to the blood flow in the blood vessels. They're both measured in millimeters of mercury (mmHg). As a general guide: high blood pressure is considered to be 140/90 mmHg or higher (or 150/90 mmHg or higher if you're over the age of 80).

In the context of the present invention the term “dyslipidemia” refers to an abnormal amount of lipids (e.g. triglycerides, cholesterol and/or fat phospholipids) in the blood. Dyslipidemia is a risk factor for the development of atherosclerotic cardiovascular disease (ASCVD), such as coronary artery disease, cerebrovascular disease, and peripheral artery disease. In developed countries, most dyslipidemias are hyperlipidemias; that is, an elevation of lipids in the blood. This is often due to poor diet and lifestyle. Prolonged elevation of insulin resistance can also lead to dyslipidemia.

In the context of the present invention the term “regular meal” refers to standard meals consumed by a subject as part of their regular daily diet, such as breakfast, lunch and dinner.

In the context of the present invention the term “pre-meal” refers to a meal consumed prior to the regular meal, such as between 1-180 minutes before consuming the regular meal.

The term ‘lean body mass’ (LBM) or ‘lean body weight’ is a part of body composition that is defined as the difference between total body weight and body fat weight. This means that it counts the mass of all organs except body fat, including bones, muscles, blood, skin, and everything else. Lean body weight is determined by dual-energy X-ray absorptiometry (DXA), which is a widely available instrument.

### DETAILED DESCRIPTION OF THE INVENTION

The present invention relates to agglomerates of soluble whey protein aggregates (AWA) for use in the treatment and/or prevention of a condition linked to GLP-1 blood levels in a patient.

### I. Agglomerates of Soluble Whey Protein Aggregates (AWA)

Agglomerates of soluble whey protein aggregates (AWA) are a special type of denatured whey protein which are prepared by denaturing native whey protein, and particularly beta-lactoglobulin, under at least partially demineralized conditions and a suitable pH to form soluble whey protein aggregates (sWPA), and subsequently agglomerating the SWPA to form the AWA.

### II. Method for Preparing AWA

In preferred embodiments of the present invention the method of producing a composition comprising AWA comprises the steps of
a) providing a whey protein solution:
   having a pH in the range of the 6-9,
   having a weight ratio between total protein and the total content of calcium and magnesium of at least 100
   comprising 1-20% w/w BLG relative to the weight of the whey protein solution
   comprising at least 30% BLG relative to total protein
b) subjecting the whey protein solution to heat-treatment which involves heating it to a temperature in the range of 68-180 degrees C. for a duration sufficient to denature at least 25% w/w of the BLG to obtain a suspension containing sWPA, and
c) subjecting the suspension or a protein concentrate thereof to processing that leads to agglomeration of the sWPA, preferably freeze-drying and preferably followed by particle size reduction of the freeze-dried product, thereby obtaining a composition comprising agglomerates of soluble whey protein aggregates.

### II.i Step a)

The whey protein solution of step a) preferably has a pH in the range of the 6-9, more preferably 6.0-9.0, even more preferably 6.3-8.7, and most preferably 6.6-8.5.

The whey protein solution of step a) preferably has a weight ratio between total protein and the total content of calcium and magnesium of at least 100, more preferably at least 150, even more preferably at least 180, and most preferably at least 190.

In preferred embodiments of the invention the whey protein solution of step a) has a weight ratio between total protein and the total content of calcium and magnesium in the range of 100-1000, more preferably 150-1000, even more preferably 180-1000, and most preferably 190-1000.

The weight ratio between total protein and the total content of calcium and magnesium is calculated as the weight of total protein divided by the sum of the weights of calcium and magnesium.

The whey protein solution of step a) preferably comprises at least 1-10% w/w BLG relative to the weight of the whey protein solution, more preferably at least 2-8% w/W, even more preferably 2-6% w/w, and most preferably 2-5% w/w BLG relative to the weight of the whey protein solution.

The whey protein solution of step a) preferably comprises at least 30% w/w BLG relative to total protein, more preferably at least 40% w/w, even more preferably at least 45% w/w, most preferably at least 50% w/w BLG relative to total protein.

The whey protein solution is preferably an aqueous solution and water typically makes up the majority of the part of the whey protein solution that it not made of solids.

The whey protein solution may contain carbohydrate, however, preferably in limited amounts. Preferably, the whey protein solution contains at most 15% w/w carbohydrate relative to the weight of the whey protein solution, more preferably at most 10% w/w, even more preferably at most 5% w/w, and most preferably at most 1% w/w carbohydrate relative to the weight of the whey protein solution.

Additionally, the whey protein solution may contain lipids, but preferably only in limited amounts. Preferably, the whey protein solution contains at most 6% w/w lipids relative to the weight of the whey protein solution, more preferably at most 2% w/w, even more preferably at most 0.5% w/w, and most preferably at most 0.1% w/w lipids relative to the weight of the whey protein solution.

The whey protein solution typically has a total solids content in the range of 1-40% w/w relative to the weight of the whey protein solution, more preferably 2-30% w/w, even more preferably 3-20% w/w and most preferably 3-10% w/w relative to the weight of the whey protein solution.

The whey protein of the whey protein solution is preferably provided by one or more of a WPC, a WPI, an SPC, and/or an SPI.

In the context of the present invention, the terms “whey protein concentrate” (WPC) and “serum protein concentrate” (SPC) pertain to dry or aqueous compositions which contain a total amount of protein of 20-89% w/w relative to total solids.

A WPC or an SPC preferably contains:
20-89% w/w protein relative to total solids,
15-70% w/w BLG relative to total protein,
8-50% w/w ALA relative to total protein, and
0-40% w/w caseinomacropeptide (CMP) relative to protein.

Alternatively, but also preferred, a WPC or an SPC may contain:
20-89% w/w protein relative to total solids,
15-90% w/w BLG relative to total protein,
4-50% w/w ALA relative to total protein, and
0-40% w/w CMP relative to protein.

Preferably, a WPC or an SPC contains:
20-89% w/w protein relative to total solids,
15-80% w/w BLG relative to total protein,
4-50% w/w ALA relative to total protein, and
0-40% w/w CMP relative to protein.

More preferably a WPC or an SPC contains:
70-89% w/w protein relative to total solids,
30-90% w/w BLG relative to total protein,
4-35% w/w ALA relative to total protein, and
0-25% w/w CMP relative to protein.

SPC typically contains no CMP or only traces of CMP.

The terms “whey protein isolate” (WPI) and “serum protein isolate” (SPI) pertain to dry or aqueous compositions which contain a total amount of protein of 90-100% w/w relative to total solids.

A WPI or an SPI preferably contains:
90-100% w/w protein relative to total solids,
15-70% w/w BLG relative to total protein,
8-50% w/w ALA relative to total protein, and
0-40% w/w CMP relative to total protein.

Alternatively, but also preferred, a WPI or an SPI may contain:
90-100% w/w protein relative to total solids,
30-95% w/w BLG relative to total protein,
4-35% w/w ALA relative to total protein, and
0-25% w/w CMP relative to total protein.

More preferably a WPI or an SPI may contain:
90-100% w/w protein relative to total solids,
30-90% w/w BLG relative to total protein,
4-35% w/w ALA relative to total protein, and
0-25% w/w CMP relative to total protein.

SPI typically contains no CMP or only traces of CMP.

The whey protein used in the whey protein solution is preferably whey protein from mammal milk, such as e.g. milk from cow, goat, mare, sheep, camel, and/or buffalo. Bovine whey protein is particularly preferred.

### II.ii Step b)

In step b), a suspension comprising sWPA is obtained by heat-treating the demineralised whey protein solution to a temperature of at least 68 degrees C. for at most 2 hours.

Preferably, the demineralised whey protein solution is heated to a temperature in the range of 68-160 degrees C., more preferably 70-145 degrees C., even more preferably 72-120 degrees C., even more preferably 73-100 degrees C., and most preferably 75-95 degrees C.

In some preferred embodiments of the invention of the duration of the heat-treatment of step b) is sufficient to denature at least 30% w/w of the BLG of the whey protein solution, more preferably at least 50% w/w, even more preferably at least 70% w/w and more preferably at least 90% w/w of the BLG of the whey protein solution.

The duration of the heat-treatment of step b) is preferably in the range of 0.1 second-120 minutes, more preferably 1 second-60 minutes, even more preferably 10 seconds-40 minutes, even more preferably 0.5 minutes-40 minutes, and most preferably 2 minutes-30 minutes.

It is furthermore preferred that step b) contains a cooling step that reduces the temperature of the obtained suspension of sWPA to at most 60 degrees C., more preferably at most 50 degrees C., even more preferably at most 20 degrees C., and most preferably at most 15 degrees C.

The inventors have found it advantageous that step b) provides sWPA in an amount of at most 10% w/w relative to the weight of the suspension, more preferably at most 8% w/w, and most preferably at most 6% w/w.

Preferably, step b) provides sWPA in an amount of 0.5-10% w/w relative to the weight of the suspension, more preferably 2-8% w/w, and most preferably 3-6% w/w.

In embodiments wherein the content of BLG and ALA of whey protein solution of step a) is the same or lower than the desired content of sWPA it is often preferred to denature as much of the BLG as possible.

However, in embodiments wherein the content of BLG and ALA of whey protein solution of step a) exceeds the desired content of sWPA it is often preferred to control the level of denaturation and hence the level of sWPA formation. This is preferably done by controlling the duration of the heat-treatment of step c). The appropriate duration is easily determined by subjecting samples of a whey protein solution to heat-treatment at a given temperature but at three different durations, quantifying the content of sWPA and plotting the resulting concentrations of formed sWPA as a function of the duration. The required duration for the desired concentration of sWPA can then be determined from the plot.

Preferably, the heat-treatment of step b) has a duration sufficient to provide sWPA in an amount of 0.5-10% w/w relative to the weight of the suspension, more preferably 2-8% w/w, and most preferably 3-6% w/w.

### II.iii Step c)

Step c) of the method involves subjecting the sWPAs obtained from step b) to conditions that lead to agglomeration of multiple sWPA.

While this can be accomplished in several ways, a presently preferred approach involves subjecting a suspension of sWPA to freeze-drying and preferably converting the obtained freeze-dried product to a powder by grinding, milling or other kinds of mechanical shear.

The sWPA-containing suspension that is subjected to freeze-drying preferably has a pH in the range of the 6-9, more preferably 6.0-9.0, even more preferably 6.3-8.7, and most preferably, 6.6-8.5.

The freeze-drying preferably involves freezing the suspension containing the sWPA to a temperature of at most-5 degrees C., more preferably at most-10 degrees C., and most preferably at most-15 degrees C.

The freeze-drying preferably involves subjecting the frozen suspension to vacuum to speed up the sublimation of water molecules, preferably at a pressure of at most 500 mBar, more preferably at most 200 mBar, even more preferably at most 50 mBar, and more preferably at most 5 mBar.

The freeze-drying process is preferably stopped when the freeze-dried product contains at most 10% w/w water, more preferably at most 8% water, even more preferably at most 6% water, and most preferably at most 5% water.

Additionally, it is often preferred to convert the product directly obtained from the freeze-drying to a powder by grinding, milling or other kinds of mechanical shearing.

Alternatively, the agglomeration may be obtained by concentrating the sWPA obtained from step b) and storing the concentrated suspension for duration sufficient for the formation of agglomerates. Addition of divalent metal ions, particularly calcium or magnesium, to the suspension or a protein concentrate thereof can provoke the formation of AWA. The AWA-containing suspensions may be used as such or subjected to further processing such as heat-treatment, optionally further concentration, and spray-drying.

In some preferred embodiments of the present invention the processing of step c. that leads to agglomeration of the sWPA involves addition of divalent metal ions, preferably calcium and/or magnesium.

While several sources of divalent metal ions can be used it is often preferred that the added divalent metal ions are provided by water-soluble salts of calcium and/or magnesium, preferably CaCl₂), MgCl₂, CaSO₄, MgSO₄, CaCO₃, MgCO₃, calcium phosphate and/or magnesium phosphate.

The divalent metal ions may be added as solid salts or in dissolved form, typically dissolved in water.

In some preferred embodiments of the present invention the divalent metal ions are added in an amount sufficient to obtain a molar ratio between the total amount of divalent metal ion and the amount of total protein of at least 1:1, more preferably at least 2:1, even more preferably at least 3:1, and most preferably at least 4:1.

By total amount of the total amount of divalent metal ion is meant to the total amount in the liquid enriched with divalent metal ions after addition of metal ions.

In other preferred embodiments of the present invention the divalent metal ions are added in an amount sufficient to obtain a molar ratio between the total amount of divalent metal ion and the amount of total protein of 1:1-100:1, more preferably 2:1-60:1, even more preferably 3:1-40:1, and more preferably 3:1-30:1.

In further preferred embodiments of the present invention the divalent metal ions are added in an amount sufficient to obtain a molar ratio between the total amount of divalent metal ion and the amount of total protein of 1:1-20:1, more preferably 2:1-16:1, even more preferably 3:1-12:1, and more preferably 3:1-10:1.

Calcium ions are particularly preferred. In some preferred embodiments of the present invention the liquid enriched with divalent metal ions has a molar percentage of calcium ions relative to the total molar content of divalent metal ions of at least 50%, more preferably at least 70% even more preferably at least 80%, and most preferably at least 90%.

The liquid enriched with divalent metal ions is the liquid obtained by addition of divalent metal ions, and optionally also by moderate pH adjustment.

Magnesium ions are typically the second most predominant type of divalent metal iona of the liquid enriched with divalent metal ions.

The compositional features and preferences described in the context of the whey protein solution of step a. equally applies to the liquid enriched with divalent metal ions, except from features relation to the content of divalent metal ions and except that a significant portion of the protein of the whey protein solution is present in denatured form in the liquid enriched with divalent metal ions.

Preferably the liquid enriched with divalent metal ions has a pH in the range of the 6-9, more preferably 6.0-9.0, even more preferably 6.3-8.7, and most preferably 6.6-8.5.

The liquid enriched with divalent metal ions is preferably mixed after the addition of the source(s) of divalent metal ions to obtain a uniform distribution of the divalent metal ions.

The liquid enriched with divalent metal ions is then subjected to an incubation step during which the sWPA agglomerate to form AWA.

In some preferred embodiments of the present invention the liquid enriched with divalent metal ions has a temperature during the incubation in the range of 10-100 degrees C., more preferably 20-90 degrees C., even more preferably 35-85 degrees C., and most preferably 40-80 degrees C.

It may for example be preferred that the liquid enriched with divalent metal ions has a temperature during the incubation in the range of 10-90 degrees C., more preferably 15-85 degrees C., even more preferably 20-85 degrees C., and most preferably 20-80 degrees C.

In some preferred embodiments of the present invention the liquid enriched with divalent metal ions is incubated for at least 1 minute, more preferably at least 2 minutes, and most preferably for at least 3 minutes.

In other preferred embodiments of the present invention the liquid enriched with divalent metal ions is incubated for at least 0.5 hour, more preferably at least 1 hour, and most preferably for at least 2 hours.

In some preferred embodiments of the present invention the liquid enriched with divalent metal ions is incubated for 1 minute to 48 hours, more preferably 2 minutes to 36 hours, and most preferably 3 minutes to 24 hours.

It is particularly preferred that the liquid enriched with divalent metal ions is incubated for 0.5 hour to 48 hours, more preferably 1 hour to 36 hours, and most preferably 2 hours to 24 hours.

It is often preferred that the liquid enriched with divalent metal ions is agitated, e.g. by stirring, during the incubation.

In some preferred embodiments of the present invention step c. furthermore involves subjecting the incubated liquid enriched with divalent metal ions to one or more of:
a concentration step, preferably one or more of evaporation, reverse osmosis, nanofiltration, ultrafiltration and/or microfiltration, and
a drying step, preferably involving spray-drying.

The drying step involve preheating the liquid to be dried to a temperature in the range of 50-80 degrees C. prior to the actual spraying that converts the liquid to be dried to droplets from which water evaporates.

In some preferred embodiments of the present invention the liquid to be dried comprises the incubated liquid enrichment with divalent metal ions. Preferably the incubated liquid enrichment with divalent metal ions makes up at least 50% w/w of the liquid to be dried, more preferably at least 70% w/w, even more preferably at least 80% w/w and most preferably at least 90% w/w.

It is often preferred that the liquid to be dried comprises the incubated liquid enrichment with divalent metal ions.

In some preferred embodiments of the present invention the liquid to be dried has a weight percentage of AWA relative to the weight of the liquid of 2-10% w/w, more preferably 3-9% w/w, and most preferably 4-8% w/w.

It is typically preferred that the liquid to be dried has a weight percentage of AWA relative to total protein of at least 40% w/w, more preferably at least 50% w/w, even more preferably at least 60% w/w, and most preferably at least 70% w/w.

It is particularly preferred that the composition comprising the AWA is prepared by a method comprising the steps of
a) providing a whey protein solution:
   having a pH in the range of the 6.6-8.5,
   having a weight ratio between total protein and the total content of calcium and magnesium of at least 180
   comprising at 2-5% w/w BLG relative to the weight of the whey protein solution
   comprising at least 40% w/w BLG relative to total protein,
b) subjecting the whey protein solution to heat-treatment which involves heating it to a temperature in the range of 75-95 degrees C. for a duration sufficient to denature at least 50% of the BLG to obtain a suspension containing sWPA, and
c) subjecting the suspension or a protein concentrate thereof to freeze-drying to prepare a freeze-dried composition followed by mechanical shearing of the of the freeze-dried product,
thereby obtaining a composition comprising agglomerates of soluble whey protein aggregates.

It is therefore particularly preferred that method of the invention comprises the steps of
a) providing a whey protein solution:
   having a pH in the range of the 6.6-8.5,
   having a weight ratio between total protein and the total content of calcium and magnesium of at least 180
   comprising at 2-5% w/w BLG relative to the weight of the whey protein solution
   comprising at least 40% w/w BLG relative to total protein,
b) subjecting the whey protein solution to heat-treatment which involves heating it to a temperature in the range of 75-95 degrees C. for a duration sufficient to denature at least 50% of the BLG to obtain a suspension containing sWPA, and
c) subjecting the suspension or a protein concentrate thereof to freeze-drying to prepare a freeze-dried composition followed by mechanical shearing of the of the freeze-dried product,
thereby obtaining a composition comprising agglomerates of soluble whey protein aggregates.

In other particularly preferred embodiments of the invention the method comprising the steps of
a) providing a whey protein solution:
   having a pH in the range of the 6.6-8.5,
   having a weight ratio between total protein and the total content of calcium and magnesium of at least 180
   comprising at 2-5% w/w BLG relative to the weight of the whey protein solution
   comprising at least 40% w/w BLG relative to total protein,
b) subjecting the whey protein solution to heat-treatment which involves heating it to a temperature in the range of 75-95 degrees C. for a duration sufficient to denature at least 50% of the BLG to obtain a suspension containing sWPA, and
c) subjecting the suspension or a protein concentrate thereof to processing that leads to agglomeration of the sWPA, which processing involves:
   addition of calcium ions and/or magnesium ions in an amount sufficient to obtain a molar ratio between the total amount of divalent metal ion and the amount of total protein of 1:1-20:1, more preferably 2:1-16:1, even more preferably 3:1-12:1, and more preferably 3:1-10:1, so that the obtained the liquid enriched with divalent metal ions has a molar percentage of calcium ions relative to the total molar content of divalent metal ions of at least 50%,
   incubating the liquid enriched with divalent metal ions at a temperature of 20-80 degrees C. for a duration of 0.5-24 h,
   optionally concentrating and/or drying the composition obtained from the incubation,
thereby obtaining a composition comprising AWA.

The inventors have found that the content of AWA provided by step c) can be calculated as the reduction in the content of sWPA caused by step c), e.g. by measuring the content of sWPA before step c) and after step c). If step b) involves drying a suspension of sWPA the quantification requires reconstitution and hydration of the dried production (e.g. for 2 hour at room temperature) before the content of sWPA is measured.

For example, in Example 1 approx. 80% of the total protein of the final freeze-dried SPI product was in the form of AWA.

On a total solids basis, the composition comprising AWA obtainable by the present method is preferably the same composition with respect to macronutrients as the whey protein solution of step a). However, the composition comprising AWA obtainable by the present method preferably contains AWA in an amount at least 20% w/w relative to total protein, more preferably at least 40% w/w, even more preferably at least 60% w/w, and more preferably at least 80% w/w relative to total protein.

The inventors have found that the AWA prepared by the method as described herein gives rise to an interesting plasma GLP-1 response when ingested by human subjects.

An aspect of the invention therefore pertains to a composition comprising AWA, which composition is obtainable according to the method defined herein, preferably in liquid or powder form.

### III. AWA for Use in Therapeutic Methods

As evidenced in the Example section, AWA provides a surprising GLP-1 response in both young and old human subjects, compared to administering placebo, but also compared to administering other whey protein compositions, such as the non-heat treated whey fraction (SPI).

It has surprisingly been found by the inventors that ingestion of AWA induces an increased and prolonged GLP-1 response as compared to ingestion of the native form of the protein as well as other protein fractions-see example 2. Consequently, the AWA structure generates an improved health benefit as compared to other whey proteins. To the inventors best knowledge this has never been reported.

In one preferred aspect, the present invention provides agglomerates of soluble whey protein aggregates (AWA) or a dosage form containing an effective amount of AWA for use as a medicament.

In one preferred embodiment, AWA or a dosage form containing an effective amount of AWA increases and/or prolongs blood levels of GLP-1 in a human subject. In one preferred embodiment, AWA or a dosage form containing an effective amount of AWA provides a delayed secretion profile of GLP-1 in the human subject. In a further preferred embodiment, AWA or a dosage form containing an effective amount of AWA (i) provides a delayed secretion profile of GLP-1, and (ii) increases and/or prolongs blood levels of GLP-1 in a human subject

In one preferred embodiment, AWA or a dosage form containing an effective amount of AWA provides a biphasic secretion profile of GLP-1 in a human subject, where a relatively small initial secretion takes place, followed by a later increased and prolonged secretion of GLP-1, measured by GLP-1 blood levels in the human subject. In a further preferred embodiment, AWA or a dosage form containing an effective amount of AWA (i) provides a biphasic secretion profile of GLP-1, and (ii) increases and/or prolongs blood levels of GLP-1 in a human subject.

Hence, the inventors have found a protein composition which induces a delayed but high GLP-1 response in a subject. Although not wishing to be bound by theory, it is speculated that AWA seem to induce a delayed gastric emptying or to be more slowly digested as compared to native whey proteins. In one preferred embodiment, the protein composition provides a delayed but high GLP-1 response in a subject when consumed as a standalone meal. In one preferred embodiment, the protein composition provides a delayed but high GLP-1 response in a subject when consumed before, during, or after a regular meal.

In one preferred aspect, the invention provides AWA or a dosage form containing an effective amount of AWA for use in treating and/or preventing one or more disorders linked to GLP-1 blood levels in a human subject.

In one preferred embodiment, the invention provides AWA or a dosage form containing an effective amount of AWA for use in treating and/or preventing one or more disorders linked to reduced GLP-1 blood levels in a human subject, compared to a healthy human subject.

In one preferred embodiment, the invention provides AWA or a dosage form containing an effective amount of AWA for use in treating and/or preventing one or more disorders where increased endogenous GLP-1 secretion could be beneficial.

In one preferred embodiment, the invention provides AWA or a dosage form containing an effective amount of AWA for use in treating and/or preventing one or more disorders linked to GLP-1 blood levels in a human subject, wherein the GLP-1 blood levels are reduced compared to a healthy human subject and where in the human subject will benefit from an increased GLP-1 blood level.

### III.i Disorders Linked to GLP-1 Blood Levels

As evidenced here, AWA affects GLP-1 levels in the blood of human subjects. Prior art describes further evidence of numerous effects of GLP-1 in the human body—as disclosed in the background section.

The inventors have here found a nutritional solution for increasing and/or prolonging the GLP-1 response, which in a human subject is most favorable for increasing the sensation of satiety and/or decreasing hunger, which may lead to a reduced food intake and/or reduce the body weight of the human subject and thereby provide a treatment for/reduce the risk of development of overweight and obesity.

In preferred embodiments, the invention provides AWA or a dosage form containing an effective amount of AWA for use in treating and/or preventing one or more disorders linked to GLP-1 blood levels in a human subject, wherein said AWA increases sensation of satiety in the human subject.

In preferred embodiments, the invention provides AWA or a dosage form containing an effective amount of AWA for use in treating and/or preventing one or more disorders linked to GLP-1 blood levels in a human subject, wherein said AWA decreases hunger in the human subject.

In preferred embodiments, the invention provides AWA or a dosage form containing an effective amount of AWA for use in treating and/or preventing one or more disorders linked to GLP-1 blood levels in a human subject, wherein said AWA reduces body weight of the human subject.

The inventors have here found a nutritional solution for increasing and/or prolonging the GLP-1 response, which in a human subject is further most favorable for improving glycemic control and/or enhancing insulin secretion and/or reducing glucagon secretion, which may lead to reducing the risk for metabolic disease and/or development of pre-diabetes and/or diabetes type-2 and associated complications.

In preferred embodiments, the invention provides AWA or a dosage form containing an effective amount of AWA for use in treating and/or preventing one or more disorders linked to GLP-1 blood levels in a human subject, wherein said AWA improves glycemic control in the human subject.

In preferred embodiments, the invention provides AWA or a dosage form containing an effective amount of AWA for use in treating and/or preventing one or more disorders linked to GLP-1 blood levels in a human subject, wherein said AWA reduces glucagon secretion in the human subject.

In preferred embodiments, the invention provides AWA or a dosage form containing an effective amount of AWA for use in treating and/or preventing one or more disorders linked to GLP-1 blood levels in a human subject, wherein said AWA enhances insulin secretion in the human subject.

The inventors have here found a nutritional solution for increasing and/or prolonging the GLP-1 response, which in a human subject is further most favorable for reducing body and/or liver fat, which may lead to reducing the risk of developing and/or amelioration of nonalcoholic fatty liver disease (NAFLD) and/or nonalcoholic steatohepatitis (NASH).

In preferred embodiments, the invention provides AWA or a dosage form containing an effective amount of AWA for use in treating and/or preventing one or more disorders linked to GLP-1 blood levels in a human subject, wherein said AWA reduces body and/or liver fat the human subject.

The inventors have here found a nutritional solution for increasing and/or prolonging the GLP-1 response, which in a human subject is further most favorable for increasing muscle microvascular perfusion thereby improving tissue oxygen, nutrient and insulin delivery and exchange, better prandial glycaemic control and tissue function and/or overcoming anabolic resistance to feeding and/or ameliorating muscle atrophy.

In preferred embodiments, the invention provides AWA or a dosage form containing an effective amount of AWA for use in treating and/or preventing one or more disorders linked to GLP-1 blood levels in a human subject, wherein said AWA increases muscle microvascular perfusion in the human subject.

In preferred embodiments, the invention provides AWA or a dosage form containing an effective amount of AWA for use in treating and/or preventing one or more disorders linked to GLP-1 blood levels in a human subject, wherein said AWA ameliorates muscle atrophy in the human subject.

The inventors have here found a nutritional solution for increasing and/or prolonging the GLP-1 response, which in a human subject is further most favorable for prevention/reduction of arteriosclerosis, hypertension, dyslipidemia, and/or cardiovascular events.

In preferred embodiments, the invention provides AWA or a dosage form containing an effective amount of AWA for use in treating and/or preventing one or more disorders linked to GLP-1 blood levels in a human subject, wherein said AWA prevents/reduces arteriosclerosis, hypertension, dyslipidemia, and/or cardiovascular events in the human subject.

In one preferred embodiment, the invention provides AWA or a dosage form containing an effective amount of AWA for use in treating and/or preventing one or more disorders linked to GLP-1 blood levels in a human subject, wherein the one or more disorders linked to GLP-1 blood levels is preferably selected from one or more of:
type II diabetes,
pre-diabetes
hyperglycemia
Nonalcoholic fatty liver disease (NAFLD)
Nonalcoholic steatohepatitis (NASH)
overweight
obesity,
obesity related disorders,
muscle atrophy
cardio vascular disease
arteriosclerosis
hypertension
dyslipidemia

In one preferred embodiment, the invention provides AWA or a dosage form containing an effective amount of AWA for use in treating and/or preventing overweight, obesity, and/or obesity related disorders.

In another preferred embodiment, the invention provides AWA or a dosage form containing an effective amount of AWA for use in treating and/or preventing type II diabetes, pre-diabetes, and/or hyperglycemia.

In another preferred embodiment, the invention provides AWA or a dosage form containing an effective amount of AWA for use in treating and/or preventing nonalcoholic fatty liver disease (NAFLD) and/or nonalcoholic steatohepatitis (NASH).

In another preferred embodiment, the invention provides AWA or a dosage form containing an effective amount of AWA for use in treating and/or preventing muscle atrophy.

In another preferred embodiment, the invention provides AWA or a dosage form containing an effective amount of AWA for use in treating and/or preventing for arteriosclerosis, hypertension, dyslipidemia, and cardiovascular events.

### III.ii Patient Group

The AWA or a dosage form containing an effective amount of AWA is preferably intended as a meal or meal supplement to a human subject. The human subject who will consume the AWA or a dosage form containing an effective amount of AWA is preferably an adult. In one preferred embodiment, the human subject is a young adult. In another preferred embodiment, the human subject is a senior. In one preferred embodiment, the human subject is an old adult. The term adult is defined as a person who is at least 18 years old. The term “young” adult is defined as an adult who is between 18 and 25 years old. The term “senior” is defined as an adult who is at least 50 years old. The term “old” adult is defined as a person who is at least 70 years old.

In one preferred embodiment, the human subject or patient group for treating and/or preventing one or more disorders linked to GLP-1 blood levels is an adult or group of adults who are at least 18, 19, 20, 21, 22, 23, 24, 25, 26, 27 28, 29, 30, 31, 32, 33, 34, 35, 36, 37, 38, 39, 40, 41, 42, 43, 44, 45, 46, 47, 48, 49, or 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 years old, or any age therein between.

The human subject may be a female or a male. In one preferred embodiment, the human subject is a male. In another preferred embodiment, the human subject is a female.

In one preferred aspect, the human subject or patient group for preventing one or more disorders linked to GLP-1 blood levels is a healthy individual(s), i.e. individuals who do not present with symptoms typical for disorders linked to GLP-1 blood levels-such as those listed in section III.i—and/or have not been diagnosed with these disorders, as will be recognized by a medical professional.

In another preferred aspect, the human subject or patient group for treating one or more disorders linked to GLP-1 blood levels is an individual(s) who presents with symptoms typical for disorders linked to GLP-1 blood levels these disorders-such as those listed in section III.i, which may or may not have not been diagnosed by a medical professional.

In one such preferred embodiment, the human subject is overweight or obese. “Overweight” is defined as having a body-mass index of at least 25.0. “Obese” is defined as having a body-mass index of at least 30.0.

In one such preferred embodiment, the human subject has high blood sugar levels, such as characterized by fasting and 2-hour postprandial blood glucose levels of more than 125 mg/dl and 180 mg/dl, respectively.

In one such preferred embodiment, the human subject has build-up of fat in the liver, such as characterized by steatosis in >5% of hepatocytes, or >5% of triglyceride of wet liver tissue weight.

In one such preferred embodiment, the human subject has abnormal amounts of lipids in the blood (dyslipidemia), such as characterized by total cholesterol level >5 mmol/l, HDL (high-density lipoproteins) level <1 mmol/l, triglycerides level >2 mmol/l, or LDL (low-density lipoproteins) level >3 mmol/l.

In one such preferred embodiment, the human subject has build-up of fatty deposits in the arteries, such as characterized by high blood pressure where the systolic pressure (higher number) and diastolic pressure (lower number) is considered to be 140/90 mmHg or higher; or 150/90 mmHg or higher if the subject is over the age of 80.

In one such preferred embodiment, the human subject has a loss of skeletal muscle mass/muscle atrophy caused by sarcopenia or cachexia or disuse, such as characterized by low muscle strength, low muscle quantity or quality or low physical performance, such as specified in Table 3 in Cruz-Jentoft et al 2019, providing recommendations for cut-off points for different parameters to increase harmonisation of sarcopenia studies.

### III.iii Administration and Dosage Regime

The AWA of the present invention is in preferred embodiments intended for oral administration to the human subject.

In preferred embodiments, AWA or a dosage form comprising AWA for use according to the invention is administered to the human subject in an amount sufficient to provide a dosage of AWA of at least 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 g/kg lean body weight per dosage.

In preferred embodiments, AWA or a dosage form comprising AWA for use according to the invention is administered to the human subject in an amount sufficient to provide a dosage of AWA of at least 0.05 g/kg lean body weight per dosage, preferably at least 0.1 g/kg lean body weight per dosage, more preferably at least 0.3 g/kg lean body weight per dosage, even more preferably at least 0.5 g/kg lean body weight per dosage.

In preferred embodiments, AWA or a dosage form comprising AWA for use according to the invention is administered to the human subject in an amount sufficient to provide a dosage of AWA of 0.05-4 g/kg lean body weight per dosage, preferably 0.05-2 g/kg lean body weight per dosage, preferably 0.1-1 g/kg lean body weight per dosage, more preferably 0.2-0.5 g/kg lean body weight per dosage.

The AWA may be administered once a day or several times a day, such as morning, midday and/or evening. In preferred embodiments AWA is administered between 1-5 times a day, such as 1, 2, 3, 4 or 5 times a day.

In one preferred embodiment, AWA or a dosage form comprising AWA for use according to the invention is administered to the human subject in an amount sufficient to provide a dosage of AWA of at least 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5 g/kg body weight per day.

In preferred embodiments, AWA or a dosage form comprising AWA for use according to the invention is administered to the human subject in an amount sufficient to provide a dosage of AWA of at least 0.05 g/kg body weight per day, preferably at least 0.1 g/kg body weight per day, more preferably at least 0.3 g/kg body weight per day, even more preferably at least 0.5 g/kg body weight per day.

In preferred embodiments, AWA or a dosage form comprising AWA for use according to the invention is administered to the human subject in an amount sufficient to provide a dosage of AWA of 0.05-4 g/kg body weight per day, preferably 0.05-2 g/kg body weight per day, preferably 0.1-1 g/kg body weight per day, more preferably 0.2-0.5 g/kg body weight per day.

In preferred embodiments, AWA or a dosage form comprising AWA for use according to the invention is administered to the human subject in an amount sufficient to provide a dosage of AWA of at least 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5 g/kg lean body weight per day.

In preferred embodiments, AWA or a dosage form comprising AWA for use according to the invention is administered to the human subject in an amount sufficient to provide a dosage of AWA of at least 0.05 g/kg lean body weight per day, preferably at least 0.1 g/kg lean body weight per day, more preferably at least 0.3 g/kg lean body weight per day, even more preferably at least 0.5 g/kg lean body weight per day.

In preferred embodiments, AWA or a dosage form comprising AWA for use according to the invention is administered to the human subject in an amount sufficient to provide a dosage of AWA of 0.05-2 g/kg lean body weight per day, preferably 0.05-1 g/kg lean body weight per day, preferably 0.1-0.5 g/kg lean body weight per day, more preferably 0.2-0.5 g/kg lean body weight per day.

The AWA or a dosage form containing an effective amount of AWA is intended as a meal or meal supplement to a human subject.

In preferred embodiments, the AWA of the present invention is administered as a standalone meal. Use of AWA as such meal may be particularly advantageous in regard to treatment and/or prevention of overweight, obesity and/or metabolic disorders

In preferred embodiments the AWA of the present invention is intended to be administered as a pre-meal.

In preferred embodiments of the invention, the AWA for use according to the invention is administered to the human subject as a pre-meal, such as administered between 5-180 minutes prior to a regular meal, between 10-180 minutes prior to a regular meal, between 15-180 minutes prior to a regular meal, between 20-180 minutes prior to a regular meal, between 25-180 minutes prior to a regular meal, between 30-180 minutes prior to a regular meal, between 35-180 minutes prior to a regular meal, between 40-180 minutes prior to a regular meal, between 45-180 minutes prior to a regular meal, between 50-180 minutes prior to a regular meal, between 55-180 minutes prior to a regular meal, between 60-180 minutes prior to a regular meal, between 75-180 minutes prior to a regular meal, between 80-180 minutes prior to a regular meal, between 85-180 minutes prior to a regular meal, between 90-180 minutes prior to a regular meal, between 95-180 minutes prior to a regular meal, between 100-180 minutes prior to a regular meal, between 105-180 minutes prior to a regular meal, between 110-180 minutes prior to a regular meal, between 115-180 minutes prior to a regular meal, between 120-180 minutes prior to a regular meal, between 125-180 minutes prior to a regular meal, between 130-180 minutes prior to a regular meal, between 135-180 minutes prior to a regular meal, between 140-180 minutes prior to a regular meal, between 145-180 minutes prior to a regular meal, between 150-180 minutes prior to a regular meal, between 155-180 minutes prior to a regular meal, between 160-180 minutes prior to a regular meal, between 165-180 minutes prior to a regular meal, between 170-180 minutes prior to a regular meal, or between 175-180 minutes prior to a regular meal.

In one preferred embodiment of the invention, the AWA for use according to the invention is administered to the human subject as a pre-meal, such as administered between 5-30 minutes prior to a regular meal, preferably between 30-60 minutes prior to a regular meal, more preferably between 60-90 minutes, preferably between 90-120 minutes, preferably between 120-150 minutes, preferably between 150-180 minutes.

Use of AWA as such a pre-meal may be particularly advantageous in regard to treatment and/or prevention of obesity/overweight to decrease hunger, thus leading to reduced food intake.

In preferred embodiments of the invention, the AWA for use according to the invention is administered to the human subject as a pre-meal, such as administered between 1-180 minutes prior to a regular meal, between 1-175 minutes prior to a regular meal, between 1-170 minutes prior to a regular meal, between 1-165 minutes prior to a regular meal, between 1-160 minutes prior to a regular meal, between 1-155 minutes prior to a regular meal, between 1-150 minutes prior to a regular meal, between 1-145 minutes prior to a regular meal, between 1-140 minutes prior to a regular meal, between 1-135 minutes prior to a regular meal, between 1-130 minutes prior to a regular meal, between 1-125 minutes prior to a regular meal, between 1-120 minutes prior to a regular meal, between 1-115 minutes prior to a regular meal, between 1-110 minutes prior to a regular meal, between 1-105 minutes prior to a regular meal, between 1-100 minutes prior to a regular meal, between 1-95 minutes prior to a regular meal, between 1-90 minutes prior to a regular meal, between 1-85 minutes prior to a regular meal, between 1-80 minutes prior to a regular meal, between 1-75 minutes prior to a regular meal, between 1-70 minutes prior to a regular meal, between 1-65 minutes prior to a regular meal, between 1-60 minutes prior to a regular meal, between 1-55 minutes prior to a regular meal, between 1-50 minutes prior to a regular meal, between 1-45 minutes prior to a regular meal, between 1-40 minutes prior to a regular meal, between 1-35 minutes prior to a regular meal, between 1-30 minutes prior to a regular meal, between 1-25 minutes prior to a regular meal, between 1-20 minutes prior to a regular meal, between 1-15 minutes prior to a regular meal, between 1-10 minutes prior to a regular meal, or between 1-5 minutes prior to a regular meal.

In one preferred embodiment of the invention, the AWA for use according to the invention is administered to the human subject as a pre-meal, such as administered between 150-180 minutes prior to a regular meal, between 120-150 minutes prior to a regular meal, between 90-120 minutes prior to a regular meal, between 60-90 minutes prior to a regular meal, preferably between 30-60 minutes prior to a regular meal, more preferably between 15-30 minutes, more preferably between 1-15 minutes.

Use of AWA as such a pre-meal may be particularly advantageous in regard to treatment and/or prevention of obesity/overweight to increase the sensation of satiety, thus leading to reduced food intake.

In preferred embodiments, the AWA of the present invention is intended to be administered in combination with a meal, such as ingested at the same time as the meal.

In one preferred embodiment of the invention, the AWA for use according to the invention is administered to the human subject in combination with a meal. Thereby, the GLP-1 response resulting from the meal adds up to the GLP-1 peak resulting from AWA which is delayed, prolonged, and increased in respect to the first peak. Thereby, the overall resulting GLP-1 response is extended and prolonged in time.

In another preferred embodiment, the AWA for use according to the invention, such as especially in regard to treatment and/or prevention of muscle atrophy, may be administered to the human subject before and/or after surgery and/or hospital stay thereby, advantageously, the subject improves his muscle mass already before undergoing surgery or a longer bedridden hospital stay and continues to maintaining such an elevated concentration of the GLP-1 during the recovery period for continued improvement of muscle mass. This provides an optimal nutritional status to minimize loss of muscle mass during the hospital intervention and also prepares him for a quicker recovery and build-up of lost muscle tissues thereafter.

The treatment or prevention provided by the present invention benefit from an additional exercise regime the an additional exercise regimen is not required for the present treatment or prevention to be effective. Hence, in one embodiment the present invention provides a treatment and/or prevention of one or more GLP-1 related disorders, which does not include an additional exercise regimen.

### Iv. A Dosage Form Comprising AWA

Those skilled in the art will understand that they can freely combine all features of the present invention disclosed herein. In particular, features described for the therapeutic use may be used and combined with the features of the dosage form of the AWA product, and vice versa.

Thus, yet a preferred aspect of the invention pertains to a dosage form as defined herein containing an effective amount of AWA.

### IV.i Type of Dosage Form

While all types of dosage forms may be used it is particularly preferred that the dosage form is selected from the group consisting of a liquid, a powder, a gel, a capsule, and/or an edible bar.

A particularly preferred dosage form is a “ready to drink beverage” (RTD), i.e. a liquid formulated for drinking as-is, preferably pasteurized or even sterilized, and packaged in a sealed container which allows for storage of the ready to drink beverage at ambient temperature or under refrigeration.

Another particularly preferred dosage form is a powder intended to be dissolved in a liquid prior to ingestion.

When the term “an effective amount of AWA” is mentioned herein in relation to a dosage form without reference to a particular disease or disorder it is preferred that the AWA is present in the dosage form in an amount sufficient to provide treat or prevent at least one of the diseases and conditions mentioned herein.

The dosage form preferably has a pH in the range of 5.5-9.0, more preferably 6.0-8.0, even more preferably 6.2-7.7, and most preferably 6.5-7.5. In another preferred embodiment, the pH of the dosage form is acidic, preferably having a pH in the range of 2-5.4, more preferably 2.5-5.0, and most preferably 3.0-4.7.

The pH values mentioned herein refer to pH values when measured according to Analysis 13 of WO 2021/136785.

### IV.ii Composition of Dosage Form

In some preferred embodiments of the present invention the dosage form comprises AWA in an amount of at least 30% w/w relative to total protein, more preferably at least 50%, even more preferably at least 60% w/w, and more preferably at least 70% w/w relative to total protein.

In some preferred embodiments of the present invention the dosage form comprises AWA in an amount of at least 4 g per intended dosage, more preferably at least 6 g per intended dosage, even more preferably at least 8 g per intended dosage, and most preferably at least 10 g per intended dosage. As disclosed previously, the AWA may be administered once a day or several times a day. The amount of AWA in the dosage form may be affected by the choice of number of dosage administrations.

In some preferred embodiments of the present invention, particularly preferred for liquid dosage forms, such as e.g. RTDs, the dosage form comprises AWA in an amount of preferably at least 1 g/100 mL, preferably at least 2 g/100 mL, preferably at least 4 g 100 mL, preferably at least 6 g 100 mL, preferably at least 8 g/100 mL, preferably at least 10 g/100 mL, preferably at least 12 g/100 mL, preferably at least 14 g/100 mL, preferably at least 16 g/100 mL, preferably at least 18 g/100 mL, preferably at least 20 g/100 mL, preferably at least 22 g/100 mL, preferably at least 24 g/100 mL, preferably at least 26 g/100 mL, preferably at least 28 g/100 mL, preferably at least 30 g/100 mL.

In some preferred embodiments of the present invention, particularly preferred for liquid dosage forms, such as e.g. RTDs, the dosage form comprises AWA in an amount of 1-20 g/100 mL, more preferably 2-18 g/100 mL, even more preferably at least 3-14 g 100 mL, and most preferably at 5-10 g 100 mL.

In some preferred embodiments of the present invention, particularly preferred for liquid dosage forms, such as e.g. RTDs, the dosage form comprises total protein in an amount of at least 1 g/100 mL, preferably at least 2 g/100 mL, preferably at least 4 g 100 mL, preferably at least 6 g 100 mL, preferably at least 8 g/100 mL, preferably at least 10 g/100 mL, preferably at least 12 g/100 mL, preferably at least 14 g/100 mL, preferably at least 16 g/100 mL, preferably at least 18 g/100 mL, preferably at least 20 g/100 mL, preferably at least 22 g/100 mL, preferably at least 24 g/100 mL, preferably at least 26 g/100 mL, preferably at least 28 g/100 mL, preferably at least 30 g 100 mL.

In some preferred embodiments of the present invention, particularly preferred for liquid dosage forms, such as e.g. RTDs, the dosage form comprising total protein in an amount of 1-25 g/100 mL, more preferably 2-20 g/100 mL, even more preferably at least 3-16 g/100 mL, and most preferably at 4-14 g/100 mL.

In some preferred embodiments of the present invention, particularly preferred for powders intended to be dissolved in a liquid prior to ingestion, such as a shake powder, the dosage form comprises AWA in an amount of at least 10 g/100 g, more preferably at least 15 g/100 g, more preferably at least 20 g/100 g, even more preferably at least 30 g/100 g, and most preferably at least 50 g/100 g.

In some preferred embodiments of the present invention, particularly preferred for powders intended to be dissolved in a liquid prior to ingestion, such as a shake powder, the dosage form comprises AWA in an amount of at least 50 g/100 g, more preferably at least 60 g/100 g, more preferably at least 70 g/100 g, even more preferably at least 80 g/100 g, and most preferably at least 90 g/100 g.

In some preferred embodiments of the present invention, particularly preferred for powders intended to be dissolved in a liquid prior to ingestion, such as a shake powder, the dosage form comprises AWA in an amount of 10-90 g/100 g, more preferably 15-85 g/100 g, even more preferably at least 20-80 g/100 g, and most preferably at 30-75 g/100 g.

The dosage forms may furthermore contain the non-protein macronutrients and micronutrients, preferably as described in pages 62-65 of WO 2021/136785 which is incorporated herein by reference.

In one preferred embodiment, the present invention provides a nutritional powder comprising
AWA in an amount of at least 30% relative to total protein, more preferably at least 50%, even more preferably at least 60% w/w, and more preferably at least 70% w/w relative to total protein, and
at least one non-dairy component.

The dosage form may comprise other macronutrients than proteins, such as e.g. carbohydrate and/or lipid.

In some embodiments of the invention, the dosage form furthermore comprises carbohydrates. The total carbohydrate content in the dosage form depends on the intended use of the dosage form.

The carbohydrate of the dosage form is preferably provided by one or more source of carbohydrate sources.

Useful carbohydrate sources may be selected from the group consisting of: sucrose, maltose, dextrose, galactose, maltodextrin, corn syrup solids, sucromalt, glucose polymers, corn syrup, modified starches, resistant starches, rice-derived carbohydrates, isomaltulose, white sugar, glucose, fructose, lactose, high fructose corn syrup, honey, sugar alcohols, fructooligosaccharides, soy fiber, corn fiber, guar gum, konjac flour, polydextrose, fibersol, and combinations thereof. In some embodiments of the invention, the dosage form comprises non-digestible sugars like fructans, the fructan comprises inulin or fructo-oligosaccharides.

In some preferred embodiments of the invention, the dosage form comprises carbohydrate between 0 to 95% of the total energy content of the dosage form, more preferably in a range between 10 to 85% of the total energy content of the dosage form, even more preferably in a range between 20 to 75% of the total energy content of the dosage form, and most preferably in a range between 30 to 60% of the total energy content of the dosage form

Even lower carbohydrate contents are often preferred, thus in some preferred embodiments of the invention the dosage form comprises carbohydrate in an amount in a range between 0 to 30% of the total energy content of the dosage form, more preferably in a range between 0 to 20% of the total energy content of the dosage form, and even more preferably in a range between 0 to 10% of the total energy content of the dosage form.

In some preferred embodiments of the present invention, the dosage form comprises a total amount of carbohydrate of at most 75% of the total energy content of the dosage form (E), more preferably at most 40 E %, even more preferably at most 10 E %, and most preferably at most 5 E %.

In some preferred embodiments of the present invention, the dosage form is particularly useful as a nutritionally incomplete nutritional supplement and comprises a total amount of carbohydrate in a range between 70-95% of the total energy content of the dosage form (E), preferably 80-90 E %.

In some preferred embodiments of the present invention, the dosage form comprises a total amount of carbohydrate in a range between 30-60% of the total energy content of the dosage form, and most preferably in a range between 35-50 E %. Such dosage form are particularly useful for nutritionally complete dosage form.

In some embodiments of the invention, the dosage form furthermore comprises at least one additional ingredient selected from the group consisting of vitamins, flavouring agent, minerals, sweeteners, antioxidants, food acid, lipids, carbohydrate, prebiotics, probiotics and non-whey protein, and a combination therefore.

The additional ingredients can be used to adjust the nutrient contribution and the taste and flavour characteristics of the dosage form.

In one embodiment of the invention, the dosage form comprises at least one high intensity sweetener (HIS). The at least one HIS is preferably selected from the group consisting of aspartame, cyclamate, sucralose, acesulfame salt, neotame, saccharin, stevia extract, a steviol glycoside such as e.g. rebaudioside A, or a combination thereof.

In some embodiments of the invention, it is particularly preferred that the sweetener comprises or even consists of one or more high intensity sweeteners.

HIS is both found among both natural and artificial sweeteners and typically have a sweetening intensity of at least 10 times that of sucrose.

If used, the total amount of HIS of the dosage form is typically in the range of 0.001-2% w/w. Preferably, the total amount of HIS is in the range of 0.005-1% w/w. Most preferably, the total amount of HIS is in the range of 0.01-0.5% w/w.

The choice of the sweetener may depend on the dosage form to be produced, e.g. high-intensity sweeteners (e.g. aspartame, acetsulfam-K or sucralose) may be used in dosage forms where no energy contribution from the sweetener is desired, whereas for dosage form having a natural profile natural sweeteners (e.g. steviol glycosides, sorbitol or sucrose) may be used.

It may furthermore be preferred that the sweetener comprises or even consists of one or more polyol sweetener(s). Non-limiting examples of useful polyol sweetener are maltitol, mannitol, lactitol, sorbitol, inositol, xylitol, threitol, galactitol or combinations thereof. If used, the total amount of polyol sweetener of the dosage form is typically in the range of 1-20% w/w. More preferably, the total amount of polyol sweetener of the dosage form is in the range of 2-15% w/w. Even more preferably, the total amount of polyol sweetener may be in the range of 4-10% w/w.

In some preferred embodiments of the invention, the dosage form comprises:
a total amount of carbohydrate of at most 1% w/w, more preferably at most 0.5% w/w, and most preferably at most 0.1% w/w, and
a total amount of HIS in the range of 0.001-2% w/w, more preferably in the range of 0.005-1% w/w, and most preferably in the range of 0.01-0.5% w/w.

In some embodiments of the invention, the dosage form furthermore comprises lipids. The total lipid content in the dosage form of the invention depends on the intended use of the dosage form.

In some preferred embodiments of the invention, the dosage form has a lipid content between 0 to 50% of the total energy content of the dosage form, more preferably in a range between 0 to 40% of the total energy content of the dosage form, even more preferably in a range between 0 to 30% of the total energy content of the dosage form, even more preferably in a range between 0 to 20% of the total energy content of the dosage form, even more preferably in a range between 0 to 10% of the total energy content of the dosage form, and most preferably in a range between 0 to 5% of the total energy content of the dosage form.

In some preferred embodiments of the present invention, the dosage form is particularly useful as a sports dosage form, i.e. a dosage form that advantageously can be ingested prior to, during, or after physical exercise, and comprises e.g. a total amount of lipid of at most 10 E %, preferably at most at most 1 E %.

In some preferred embodiments of the present invention, the dosage form is particularly useful as a nutritionally incomplete nutritional supplement and comprises a total amount of lipid of at most 10% of the total energy content of the dosage form, preferably at most at most 1 E %.

In some preferred embodiments of the present invention, the dosage form, e.g. in the form of a sports dosage form, comprises:
a total amount of protein in the range of 1-30% w/w relative to the weight of the dosage form, more preferably 4-24% w/w relative to the weight of the dosage form, even more preferably 8-20% w/w relative to the weight of the dosage form, and most preferably 10-20% w/w relative to the weight of the dosage form,
a total amount of carbohydrate of at most 75% of the total energy content of the dosage form (E), more preferably at most 40 E %, even more preferably at most 10 E %, and most preferably at most 5 E %, and
a total amount of lipid of at most 10 E %, more preferably at most 6 E %, even more preferably at most 3 E %, and most preferably at most 1 E %.

In other preferred embodiments of the present invention, the dosage form, e.g. in the form of a low carbohydrate sports dosage form, comprises:
a total amount of protein in the range of 1-30% w/w relative to the weight of the dosage form, more preferably 4-24% w/w relative to the weight of the dosage form, even more preferably 8-20% w/w relative to the weight of the dosage form, and most preferably 10-20% w/w relative to the weight of the dosage form,
a total amount of carbohydrate of at most 10 E %, more preferably at most 6 E %, even more preferably at most 3 E %, and most preferably at most 1 E %,
a total amount of lipid of at most 5 E %, more preferably at most 4 E %, even more preferably at most 3 E %, and most preferably at most 1 E %, and
a total amount of HIS in the range of 0.001-2% w/w, more preferably in the range of 0.005-1% w/w, and most preferably in the range of 0.01-0.5% w/w.

In other preferred embodiments of the present invention, the dosage form, e.g. in the form of a nutritionally complete dosage form, comprises:
a total amount of protein in the range of 1-30% w/w relative to the weight of the dosage form, more preferably 4-24% w/w relative to the weight of the dosage form, even more preferably 8-20% w/w relative to the weight of the dosage form, and most preferably 10-20% w/w relative to the weight of the dosage form,
a total amount of carbohydrate in a range between 30-60% of the total energy content of the dosage form, and most preferably in a range between 35-50 E % and
a total amount of lipid in the range of 20-50% of the total energy content, more preferably in a range between 25-45 E %, and most preferably 30-40 E %.

In some preferred embodiments of the invention, the AWA used to prepare the dosage form is provided by an AWA-containing composition obtainable by the method described herein.

Preferably, the preparation of the dosage form involves mixing an AWA-containing composition obtainable by the method described herein with one or more further ingredients required for the dosage form.

The dosage form of the present invention may advantageously be used as a pre-meal.

### v. Method of Treatment

In one preferred aspect, the present invention provides a method for treatment and/or prevention of one or more disorders linked to GLP-1 blood levels, wherein said method comprises administration of AWA or a dosage form containing an effective amount of AWA to a human subject.

In preferred embodiments, the present invention provides a method for treatment and/or prevention of one or more disorders linked to GLP-1 blood levels, wherein said method comprises administration of AWA or a dosage form containing an effective amount of AWA to a human subject, and wherein increasing the GPL-1 levels is beneficial in the treatment/prevention of said disorder.

In preferred embodiments, the present invention provides a method for treatment and/or prevention of one or more disorders linked to GLP-1 blood levels, wherein said method comprises administration of AWA or a dosage form containing an effective amount of AWA to a human subject, wherein said AWA increases the sensation of satiety/decreases hunger in the human subject.

In preferred embodiments, the present invention provides a method for treatment and/or prevention of one or more disorders linked to GLP-1 blood levels, wherein said method comprises administration of AWA or a dosage form containing an effective amount of AWA to a human subject, wherein said AWA enhances insulin secretion, reduces glucagon secretion and/or improves glycemic control in the human subject.

In preferred embodiments, the present invention provides a method for treatment and/or prevention of one or more disorders linked to GLP-1 blood levels, wherein said method comprises administration of AWA or a dosage form containing an effective amount of AWA to a human subject, wherein said AWA reduces body weight of the human subject.

In preferred embodiments, the present invention provides a method for treatment and/or prevention of one or more disorders linked to GLP-1 blood levels, wherein said method comprises administration of AWA or a dosage form containing an effective amount of AWA to a human subject, wherein said AWA reduces body and/or liver fat of the human subject.

In preferred embodiments, the present invention provides a method for treatment and/or prevention of one or more disorders linked to GLP-1 blood levels, wherein said method comprises administration of AWA or a dosage form containing an effective amount of AWA to a human subject, wherein said AWA ameliorates muscle atrophy in the human subject.

In preferred embodiments, the present invention provides a method for treatment and/or prevention of one or more disorders linked to GLP-1 blood levels, wherein said method comprises administration of AWA or a dosage form containing an effective amount of AWA to a human subject, wherein said AWA reduces arteriosclerosis, hypertension, dyslipidemia, and/or cardiovascular events.

In preferred embodiments, the present invention provides a method for treatment and/or prevention of one or more disorders selected from the list: type II diabetes, pre-diabetes, hyperglycemia, Nonalcoholic fatty liver disease (NAFLD), Nonalcoholic steatohepatitis (NASH), overweight, obesity, obesity related disorders, muscle atrophy, cardio vascular disease, arteriosclerosis, hypertension and dyslipidemia; wherein said method comprises administration of AWA or a dosage form containing an effective amount of AWA to a human subject.

In preferred embodiments, the present invention provides a method for treatment and/or prevention of overweight, obesity, and/or obesity related disorders; wherein said method comprises administration of AWA or a dosage form containing an effective amount of AWA to a human subject.

In preferred embodiments, the present invention provides a method for treatment and/or prevention of type II diabetes, pre-diabetes, and/or hyperglycemia; wherein said method comprises administration of AWA or a dosage form containing an effective amount of AWA to a human subject.

In preferred embodiments, the present invention provides a method for treatment and/or prevention of nonalcoholic fatty liver disease (NAFLD) and/or nonalcoholic steatohepatitis (NASH); wherein said method comprises administration of AWA or a dosage form containing an effective amount of AWA to a human subject.

In preferred embodiments, the present invention provides a method for treatment and/or prevention of muscle atrophy; wherein said method comprises administration of AWA or a dosage form containing an effective amount of AWA to a human subject.

In preferred embodiments, the present invention provides a method for treatment and/or prevention of arteriosclerosis, hypertension, dyslipidemia, and cardiovascular events; wherein said method comprises administration of AWA or a dosage form containing an effective amount of AWA to a human subject.

### VI. Non-Therapeutic Methods of Treatment

In one preferred aspect, the invention provides a method, preferably non-therapeutic method, for increasing and/or prolonging the secretion of glucagon-like peptide 1 (GLP-1) in a human subject, wherein said method comprises administering to the human subject an effective amount of AWA or a dosage form containing an effective amount of AWA.

In preferred embodiments, the invention provides a method, preferably non-therapeutic method, for decreasing hunger and/or increasing sensation of satiety in a human subject, wherein said method comprises administering to the human subject an effective amount of AWA or a dosage form containing an effective amount of AWA.

In preferred embodiments, the invention provides a method, preferably non-therapeutic method, for reducing body weight of a human subject, wherein said method comprises administering to the human subject an effective amount of AWA or a dosage form containing an effective amount of AWA.

In preferred embodiments, the invention provides a method, preferably non-therapeutic method, for decreasing body and/or liver fat in a human subject, wherein said method comprises administering to the human subject an effective amount of AWA or a dosage form containing an effective amount of AWA.

In preferred embodiments, the invention provides a method, preferably non-therapeutic method, for enhancing insulin secretion, reducing glucagon secretion, and/or improving glycemic control in a human subject, wherein said method comprises administering to the human subject an effective amount of AWA or a dosage form containing an effective amount of AWA.

In preferred embodiments, the invention provides a method, preferably non-therapeutic method, for ameliorating muscle atrophy, wherein said method comprises administering to the human subject an effective amount of AWA or a dosage form containing an effective amount of AWA.

In preferred embodiments, the invention provides a method, preferably non-therapeutic method, for reducing arteriosclerosis, hypertension, dyslipidemia, and/or cardiovascular events, wherein said method comprises administering to the human subject an effective amount of AWA or a dosage form containing an effective amount of AWA.

### VII. Use of AWA

In one preferred aspect, the invention concerns the use of an effective amount of AWA or a dosage form containing an effective amount of AWA for increasing and/or prolonging the secretion of glucagon-like peptide 1 (GLP-1) in a human subject.

In preferred embodiments, the invention concerns the use of AWA or a dosage form containing an effective amount of AWA for decreasing hunger and/or increasing sensation of satiety in a human subject.

In preferred embodiments, the invention concerns the use AWA or a dosage form containing an effective amount of AWA for decreasing body and/or liver fat in a human subject.

In preferred embodiments, the invention concerns the use AWA or a dosage form containing an effective amount of AWA for reducing body weight of a human subject.

In preferred embodiments, the invention concerns the use AWA or a dosage form containing an effective amount of AWA for enhancing insulin secretion, reducing glucagon secretion, and/or improving glycemic control in a human subject.

In preferred embodiments, the invention concerns the use AWA or a dosage form containing an effective amount of AWA for ameliorating muscle atrophy in a human subject.

In preferred embodiments, the invention concerns the use AWA or a dosage form containing an effective amount of AWA for reducing arteriosclerosis, hypertension, dyslipidemia, and/or cardiovascular events

### VIII. Use of AWA in the Manufacture of a Medicament

In one preferred aspect, the present invention concerns the use of AWA or a dosage form containing an effective amount of AWA in the manufacture of a medicament for the treatment and/or prevention of one or more of
type II diabetes,
pre-diabetes
hyperglycemia
Nonalcoholic fatty liver disease (NAFLD)
Nonalcoholic steatohepatitis (NASH)
overweight
obesity,
obesity related disorders,
muscle atrophy
cardio vascular disease
arteriosclerosis
hypertension
dyslipidemia

### Preferred Numbered Embodiments

In the following, preferred numbered embodiments of the invention are described.

Preferred numbered embodiment 1. Agglomerates of soluble whey protein aggregates (AWA) or a dosage form containing an effective amount of AWA for use as a medicament.

Preferred numbered embodiment 2. The AWA or the dosage form containing an effective amount of AWA according to any of the preceding preferred numbered embodiments, for use in treating and/or preventing one or more disorders linked to GLP-1 blood levels in a human subject.

Preferred numbered embodiment 3. The AWA or the dosage form containing an effective amount of AWA for use in treating and/or preventing according to any of the preceding preferred numbered embodiments, wherein the AWA increases and/or prolongs blood levels of GLP-1 in the human subject.

Preferred numbered embodiment 4. The AWA or the dosage form containing an effective amount of AWA for use in treating and/or preventing according to any of the preceding preferred numbered embodiments, wherein the AWA provides a biphasic secretion profile of GLP-1 in the human subject.

Preferred numbered embodiment 5. The AWA or the dosage form containing an effective amount of AWA for use in treating and/or preventing according to any of the preceding preferred numbered embodiments, wherein the AWA provides a delayed secretion profile of GLP-1 in the human subject.

Preferred numbered embodiment 6. The AWA or the dosage form containing an effective amount of AWA for use in treating and/or preventing according to any of the preceding preferred numbered embodiments, wherein said AWA increases the sensation of satiety of the human subject.

Preferred numbered embodiment 7. The AWA or the dosage form containing an effective amount of AWA for use in treating and/or preventing according to any of the preceding preferred numbered embodiments, wherein said AWA decreases hunger of the human subject.

Preferred numbered embodiment 8. The AWA or the dosage form containing an effective amount of AWA for use in treating and/or preventing according to any of the preceding preferred numbered embodiments, wherein said AWA reduces body weight of the human subject.

Preferred numbered embodiment 9. The AWA or the dosage form containing an effective amount of AWA for use in treating and/or preventing according to any of the preceding preferred numbered embodiments, wherein said AWA improves glycemic control of the human subject.

Preferred numbered embodiment 10. The AWA or the dosage form containing an effective amount of AWA for use in treating and/or preventing according to any of the preceding preferred numbered embodiments, wherein said AWA enhances insulin secretion of the human subject.

Preferred numbered embodiment 11. The AWA or the dosage form containing an effective amount of AWA for use in treating and/or preventing according to any of the preceding preferred numbered embodiments, wherein said AWA reduces glucagon secretion of the human subject.

Preferred numbered embodiment 12. The AWA or the dosage form containing an effective amount of AWA for use in treating and/or preventing according to any of the preceding preferred numbered embodiments, wherein said AWA reduces body and/or liver fat of the human subject.

Preferred numbered embodiment 13. The AWA or the dosage form containing an effective amount of AWA for use in treating and/or preventing according to any of the preceding preferred numbered embodiments, wherein said AWA ameliorate muscle atrophy of the human subject.

Preferred numbered embodiment 14. The AWA or the dosage form containing an effective amount of AWA for use in treating and/or preventing according to any of the preceding preferred numbered embodiments, wherein said AWA reduces risk of arteriosclerosis, hypertension, dyslipidemia, and/or cardiovascular events of the human subject.

Preferred numbered embodiment 15. The AWA or the dosage form containing an effective amount of AWA, for use in treatment and/or prevention according to any of the preceding preferred numbered embodiments, wherein said disorders linked to GLP-1 blood levels are selected from one or more of:
a. type II diabetes,
b. pre-diabetes
c. hyperglycemia
d. Nonalcoholic fatty liver disease (NAFLD)
e. Nonalcoholic steatohepatitis (NASH)
f. overweight
g. obesity,
h. obesity related disorders,
i. muscle atrophy
j. cardio vascular disease
k. arteriosclerosis
l. hypertension
m. dyslipidemia

Preferred numbered embodiment 16. The AWA or the dosage form containing an effective amount of AWA for use in treatment and/or prevention according to any of the preceding preferred numbered embodiments, wherein the human subject is an adult, and more preferably a senior.

Preferred numbered embodiment 17. The AWA or the dosage form containing an effective amount of AWA for use in treatment and/or prevention according to any of the preceding preferred numbered embodiments, wherein the human subject is a young adult.

Preferred numbered embodiment 18. The AWA or the dosage form containing an effective amount of AWA for use in treatment and/or prevention according to any of the preceding preferred numbered embodiments, wherein the human subject is a healthy individual.

Preferred numbered embodiment 19. The AWA or the dosage form containing an effective amount of AWA for use in treatment and/or prevention according to any of the preceding preferred numbered embodiments, wherein the human subject is overweight and more preferably obese.

Preferred numbered embodiment 20. The AWA or the dosage form containing an effective amount of AWA for use in treatment and/or prevention according to any of the preceding preferred numbered embodiments, wherein said AWA or said dosage form is for oral administration to the human subject.

Preferred numbered embodiment 21. The AWA or the dosage form containing an effective amount of AWA, for use in treatment and/or prevention according to any of the preceding preferred numbered embodiments, wherein said AWA or said dosage form is administered in combination with a meal, preferably as a pre-meal, between 5-30 mins prior to a regular meal, preferably between 30-60 minutes prior to a regular meal, more preferably between 60-90 minutes, preferably between 90-120 minutes, preferably between 120-150 minutes, preferably between 150-180 minutes.

Preferred numbered embodiment 22. The AWA or the dosage form containing an effective amount of AWA, for use in treatment and/or prevention according to any of the preceding preferred numbered embodiments, wherein the AWA or the dosage form is administered as a pre-meal, such as between 1-180 minutes prior to a regular meal.

Preferred numbered embodiment 23. The AWA or the dosage form containing an effective amount of AWA, for use in treatment and/or prevention according to any one of the preceding preferred numbered embodiments, wherein the AWA or the dosage form containing an effective amount of AWA is administered in an amount sufficient to provide a dosage of AWA of at least 0.05 g/kg lean body weight per dosage, preferably at least 0.1 g/kg lean body weight per dosage, more preferably at least 0.3 g/kg lean body weight per dosage, even more preferably at least 0.5 g/kg lean body weight per dosage.

Preferred numbered embodiment 24. The AWA or the dosage form containing an effective amount of AWA, for use in treatment and/or prevention according to any one of the preceding preferred numbered embodiments, wherein the AWA or the dosage form containing an effective amount of AWA is administered in an amount sufficient to provide a dosage of AWA of at least 0.05 g/kg lean body weight per dosage, preferably at least 0.1 g/kg lean body weight per dosage, more preferably at least 0.3 g/kg lean body weight per dosage, even more preferably at least 0.5 g/kg lean body weight per dosage, wherein said dosage is administered between 1-5 times a day, such as 1, 2, 3, 4 or 5 times a day.

Preferred numbered embodiment 25. The AWA or the dosage form for use in treatment and/or prevention according to any of the preceding preferred numbered embodiments, wherein said AWA or said dosage form is administered in an amount sufficient to provide a dosage of AWA of 0.05-4 g/kg body weight per day, preferably 0.05-2 g/kg body weight per day, preferably 0.1-1 g/kg body weight per day, more preferably 0.2-0.5 g/kg body weight per day.

Preferred numbered embodiment 26. The dosage form for use in treatment and/or prevention according to any of the preceding preferred numbered embodiments, wherein the dosage form is selected from the group consisting of a liquid, a powder, a gel, a capsule, and edible bar.

Preferred numbered embodiment 27. The dosage form for use in treatment and/or prevention according to any of the preceding preferred numbered embodiments, wherein the dosage form is a liquid, wherein the liquid is a “ready to drink beverage”.

Preferred numbered embodiment 28. The dosage form for use in treatment and/or prevention according to any of the preceding preferred numbered embodiments, wherein the dosage form is a powder intended to be dissolved in a liquid prior to ingestion.

Preferred numbered embodiment 29. The dosage form for use in treatment and/or prevention according to any of the preceding preferred numbered embodiments, comprising AWA in an amount of at least 30% w/w relative to total protein, more preferably at least 50%, even more preferably at least 60% w/w, and more preferably at least 70% w/w relative to total protein.

Preferred numbered embodiment 30. The dosage form for use in treatment and/or prevention according to any of the preceding preferred numbered embodiments, comprising AWA in an amount of at least 4 g per intended dosage, more preferably at least 6 g per intended dosage, even more preferably at least 8 g per intended dosage, and most preferably at least 10 g per intended dosage.

Preferred numbered embodiment 31. The dosage form for use in treatment and/or prevention according to any of the preceding preferred numbered embodiments, comprising AWA in an amount of at least 1 g/100 mL, preferably at least 2 g/100 mL, preferably at least 4 g 100 mL, preferably at least 6 g 100 mL, preferably at least 8 g/100 mL, preferably at least 10 g/100 mL, preferably at least 12 g/100 mL, preferably at least 14 g/100 mL, preferably at least 16 g/100 mL, preferably at least 18 g/100 mL, preferably at least 20 g/100 mL, preferably at least 22 g/100 mL, preferably at least 24 g/100 mL, preferably at least 26 g/100 mL, preferably at least 28 g/100 mL, preferably at least 30 g/100 mL.

Preferred numbered embodiment 32. The dosage form for use in treatment and/or prevention according to any of the preceding preferred numbered embodiments, comprising AWA in an amount of 1-20 g/100 mL, more preferably 2-18 g/100 mL, even more preferably at least 3-14 g 100 mL, and most preferably at 5-10 g 100 mL.

Preferred numbered embodiment 33. The dosage form for use in treatment and/or prevention according to any of the preceding preferred numbered embodiments, comprising total protein in an amount of at least 1 g/100 mL, preferably at least 2 g/100 mL, preferably at least 4 g 100 mL, preferably at least 6 g 100 mL, preferably at least 8 g/100 mL, preferably at least 10 g/100 mL, preferably at least 12 g/100 mL, preferably at least 14 g/100 mL, preferably at least 16 g/100 mL, preferably at least 18 g/100 mL, preferably at least 20 g/100 mL, preferably at least 22 g/100 mL, preferably at least 24 g/100 mL, preferably at least 26 g/100 mL, preferably at least 28 g/100 mL, preferably at least 30 g 100 mL.

Preferred numbered embodiment 34. The dosage form for use in treatment and/or prevention according to any of the preceding preferred numbered embodiments, comprising total protein in an amount of 1-25 g/100 mL, more preferably 2-20 g/100 mL, even more preferably at least 3-16 g/100 mL, and most preferably at 4-14 g/100 mL.

Preferred numbered embodiment 35. The dosage form for use in treatment and/or prevention according to any of the preceding preferred numbered embodiments, comprising AWA in an amount of at least 10 g/100 g, more preferably at least 15 g/100 g, more preferably at least 20 g/100 g, even more preferably at least 30 g/100 g, and most preferably at least 50 g/100 g.

Preferred numbered embodiment 36. The dosage form for use in treatment and/or prevention according to any of the preceding preferred numbered embodiments, comprising AWA in an amount of at least 50 g/100 g, more preferably at least 60 g/100 g, more preferably at least 70 g/100 g, even more preferably at least 80 g/100 g, and most preferably at least 90 g/100 g.

Preferred numbered embodiment 37. The dosage form for use in treatment and/or prevention according to any of the preceding preferred numbered embodiments, comprising AWA in an amount of 10-90 g/100 g, more preferably 15-85 g/100 g, even more preferably at least 20-80 g/100 g, and most preferably at 30-75 g/100 g.

Preferred numbered embodiment 38. The dosage form for use in treatment and/or prevention according to any of the preceding preferred numbered embodiments, having a pH in the range of 5.5-9.0, more preferably 6.0-8.0, even more preferably 6.2-7.7, and most preferably 6.5-7.5.

Preferred numbered embodiment 39. The dosage form for use in treatment and/or prevention according to any of the preceding preferred numbered embodiments, having an acidic pH, preferably having a pH in the range of 2-5.4, more preferably 2.5-5.0, and most preferably 3.0-4.7.

Preferred numbered embodiment 40. The dosage form for use in treatment and/or prevention according to any of the preceding preferred numbered embodiments, wherein the dosage form additionally comprises least one non-dairy component.

Preferred numbered embodiment 41. A nutritional powder comprising
AWA in an amount of at least 30% w/w relative to total protein, more preferably at least 50%, even more preferably at least 60% w/w, and more preferably at least 70% w/w relative to total protein, and
at least one non-dairy component.

Preferred numbered embodiment 42. The AWA or the dosage form containing an effective amount of AWA for use in treatment and/or prevention according to any of the preceding preferred numbered embodiments, or the nutritional powder comprising AWA according to any of the preceding preferred numbered embodiments, wherein the protein of the AWA comprises beta-lactoglobulin (BLG) in an amount of at least 50% w/w, more preferably at least 70% w/w even more preferably at least 80% w/w and more preferably at least 90% w/w.

Preferred numbered embodiment 43. The AWA or the dosage form containing an effective amount of AWA for use in treatment and/or prevention according to any of the preceding preferred numbered embodiments, or the nutritional powder comprising AWA according to any of the preceding preferred numbered embodiments, wherein the AWA is obtainable by, and e.g. obtained by,
a. providing a whey protein solution:
   having a pH in the range of the 6-9,
   having a weight ratio between total protein and the total content of calcium and magnesium of at least 100
   comprising at least 1-10% w/w BLG relative to the weight of the whey protein solution
   comprising at least 30% BLG relative to total protein
b. subjecting the whey protein solution to heat-treatment which involves heating it to a temperature in the range of 68-180 degrees C. for a duration sufficient to denature at least 25% w/w of the BLG to obtain a suspension containing sWPA, and
c. subjecting the suspension or a protein concentrate thereof to processing that leads to agglomeration of the sWPA, preferably freeze-drying followed by particle size reduction, thereby obtaining agglomerates of soluble whey protein aggregates.

Preferred numbered embodiment 44. A method of producing a composition comprising AWA, comprising the steps of
a. providing a whey protein solution:
   having a pH in the range of the 6-9,
   having a weight ratio between total protein and the total content of calcium and magnesium of at least 100
   comprising at least 1-10% w/w BLG relative to the weight of the whey protein solution
   comprising at least 30% BLG relative to total protein
b. subjecting the whey protein solution to heat-treatment which involves heating it to a temperature in the range of 68-180 degrees C. for a duration sufficient to denature at least 25% w/w of the BLG to obtain a suspension containing sWPA, and
c. subjecting the suspension or a protein concentrate thereof to processing that leads to agglomeration of the sWPA, preferably freeze-drying and preferably followed by particle size reduction of the freeze-dried product,
thereby obtaining a composition comprising agglomerates of soluble whey protein aggregates.

Preferred numbered embodiment 45. A method for treatment and/or prevention of one or more disorders linked to GLP-1 blood levels, wherein said method comprises administration of AWA or a dosage form containing an effective amount of AWA to a human subject.

Preferred numbered embodiment 46. The method for treatment and/or prevention according to any preceding preferred numbered embodiment, wherein said disorder is selected from one or more of
a. type II diabetes,
b. pre-diabetes
c. hyperglycemia
d. Nonalcoholic fatty liver disease (NAFLD)
e. Nonalcoholic steatohepatitis (NASH)
f. overweight
g. obesity,
h. obesity related disorders,
i. muscle atrophy
j. cardio vascular disease
k. arteriosclerosis
l. hypertension
m. dyslipidemia

Preferred numbered embodiment 47. The method for treatment and/or prevention according to any preceding preferred numbered embodiment, wherein said AWA
a. increases the sensation of satiety
b. decreases hunger
c. reduces body weight
d. improves glycemic control
e. reduces glucagon seretion
f. enhances insulin secretion
g. reduces body and/or liver fat
h. ameliorates muscle atrophy, and/or
i. reduces risk of arteriosclerosis, hypertension, dyslipidemia, and/or cardiovascular events
in a human subject.

Preferred numbered embodiment 48. The method for treatment and/or prevention according to any of the preceding preferred numbered embodiments, wherein the human subject is an adult, and more preferably a senior.

Preferred numbered embodiment 49. The method for treatment and/or prevention according to any of the preceding preferred numbered embodiments, wherein the human subject is a young adult.

Preferred numbered embodiment 50. The method for treatment and/or prevention according to any of the preceding preferred numbered embodiments, wherein the human subject is overweight and more preferably obese.

Preferred numbered embodiment 51. The method for treatment and/or prevention according to any of the preceding preferred numbered embodiments, wherein said AWA or said dosage form is administered by oral administration.

Preferred numbered embodiment 52. The method for treatment and/or prevention according to any of the preceding preferred numbered embodiments, wherein said AWA or said dosage form is administered in combination with a meal, preferably as a pre-meal, between 5-30 mins prior to a regular meal, preferably between 30-60 minutes prior to a regular meal, more preferably between 60-90 minutes, preferably between 90-120 minutes, preferably between 120-150 minutes, preferably between 150-180 minutes . . .

Preferred numbered embodiment 53. The method for treatment and/or prevention according to any of the preceding preferred numbered embodiments, wherein the AWA or the dosage form is administered as a pre-meal, such as between 1-180 minutes prior to a regular meal.

Preferred numbered embodiment 54. The method for treatment and/or prevention according to any of the preceding preferred numbered embodiments, wherein the AWA or the dosage form containing an effective amount of AWA is administered in an amount sufficient to provide a dosage of AWA of at least 0.05 g/kg lean body weight per dosage, preferably at least 0.1 g/kg lean body weight per dosage, more preferably at least 0.3 g/kg lean body weight per dosage, even more preferably at least 0.5 g/kg lean body weight per dosage.

Preferred numbered embodiment 55. The method for treatment and/or prevention according to any of the preceding preferred numbered embodiments, wherein the AWA or the dosage form containing an effective amount of AWA is administered in an amount sufficient to provide a dosage of AWA of at least 0.05 g/kg lean body weight per dosage, preferably at least 0.1 g/kg lean body weight per dosage, more preferably at least 0.3 g/kg lean body weight per dosage, even more preferably at least 0.5 g/kg lean body weight per dosage, wherein said dosage is administered between 1-5 times a day, such as 1, 2, 3, 4 or 5 times a day.

Preferred numbered embodiment 56. The method for treatment and/or prevention according to any of the preceding preferred numbered embodiments, wherein said AWA or said dosage form is administered in an amount sufficient to provide a dosage of AWA of 0.05-4 g/kg body weight per day, preferably 0.05-2 g/kg body weight per day, preferably 0.1-1 g/kg body weight per day, more preferably 0.2-0.5 g/kg body weight per day.

Preferred numbered embodiment 57. The method for treatment and/or prevention according to any of the preceding preferred numbered embodiments, wherein the dosage form is selected from the group consisting of a liquid, a powder, a gel, a capsule, and edible bar.

Preferred numbered embodiment 58. The method for treatment and/or prevention according to any of the preceding preferred numbered embodiments, wherein the dosage form is a liquid, wherein the liquid is a “ready to drink beverage”.

Preferred numbered embodiment 59. The method for treatment and/or prevention according to any of the preceding preferred numbered embodiments, wherein the dosage form is a powder intended to be dissolved in a liquid prior to ingestion.

Preferred numbered embodiment 60. The method for treatment and/or prevention according to any of the preceding preferred numbered embodiments, wherein the dosage form comprises AWA in an amount of at least 30% w/w relative to total protein, more preferably at least 50%, even more preferably at least 60% w/w, and more preferably at least 70% w/w relative to total protein.

Preferred numbered embodiment 61. The method for treatment and/or prevention according to any of the preceding preferred numbered embodiments, wherein the dosage form comprises AWA in an amount of at least 4 g per intended dosage, more preferably at least 6 g per intended dosage, even more preferably at least 8 g per intended dosage, and most preferably at least 10 g per intended dosage.

Preferred numbered embodiment 62. The method for treatment and/or prevention according to any of the preceding preferred numbered embodiments, wherein the dosage form comprises AWA in an amount of at least 1 g/100 mL, preferably at least 2 g/100 mL, preferably at least 4 g 100 mL, preferably at least 6 g 100 mL, preferably at least 8 g/100 mL, preferably at least 10 g/100 mL, preferably at least 12 g/100 mL, preferably at least 14 g/100 mL, preferably at least 16 g/100 mL, preferably at least 18 g/100 mL, preferably at least 20 g/100 mL, preferably at least 22 g/100 mL, preferably at least 24 g/100 mL, preferably at least 26 g/100 mL, preferably at least 28 g/100 mL, preferably at least 30 g/100 mL.

Preferred numbered embodiment 63. The method for treatment and/or prevention according to any of the preceding preferred numbered embodiments, wherein the dosage form comprises AWA in an amount of 1-20 g/100 mL, more preferably 2-18 g/100 mL, even more preferably at least 3-14 g 100 mL, and most preferably at 5-10 g 100 mL.

Preferred numbered embodiment 64. The method for treatment and/or prevention according to any of the preceding preferred numbered embodiments, wherein the dosage form comprises total protein in an amount of at least 1 g/100 mL, preferably at least 2 g/100 mL, preferably at least 4 g 100 mL, preferably at least 6 g 100 mL, preferably at least 8 g/100 mL, preferably at least 10 g/100 mL, preferably at least 12 g/100 mL, preferably at least 14 g/100 mL, preferably at least 16 g/100 mL, preferably at least 18 g/100 mL, preferably at least 20 g/100 mL, preferably at least 22 g/100 mL, preferably at least 24 g/100 mL, preferably at least 26 g/100 mL, preferably at least 28 g/100 mL, preferably at least 30 g 100 mL.

Preferred numbered embodiment 65. The method for treatment and/or prevention according to any of the preceding preferred numbered embodiments, wherein the dosage form comprises total protein in an amount of 1-25 g/100 mL, more preferably 2-20 g/100 mL, even more preferably at least 3-16 g/100 mL, and most preferably at 4-14 g/100 mL.

Preferred numbered embodiment 66. The method for treatment and/or prevention according to any of the preceding preferred numbered embodiments, wherein the dosage form comprises AWA in an amount of at least 10 g/100 g, more preferably at least 15 g/100 g, more preferably at least 20 g/100 g, even more preferably at least 30 g/100 g, and most preferably at least 50 g/100 g.

Preferred numbered embodiment 67. The method for treatment and/or prevention according to any of the preceding preferred numbered embodiments, wherein the dosage form comprises AWA in an amount of at least 50 g/100 g, more preferably at least 60 g/100 g, more preferably at least 70 g/100 g, even more preferably at least 80 g/100 g, and most preferably at least 90 g/100 g.

Preferred numbered embodiment 68. The method for treatment and/or prevention according to any of the preceding preferred numbered embodiments, wherein the dosage form comprises AWA in an amount of 10-90 g/100 g, more preferably 15-85 g/100 g, even more preferably at least 20-80 g/100 g, and most preferably at 30-75 g/100 g.

Preferred numbered embodiment 69. The method for treatment and/or prevention according to any of the preceding preferred numbered embodiments, wherein pH of the dosage form is in the range of 5.5-9.0, more preferably 6.0-8.0, even more preferably 6.2-7.7, and most preferably 6.5-7.5.

Preferred numbered embodiment 70. The method for treatment and/or prevention according to any of the preceding preferred numbered embodiments, wherein pH of the dosage form is acidic, preferably in the range of 2-5.4, more preferably 2.5-5.0, and most preferably 3.0-4.7.

Preferred numbered embodiment 71. The method for treatment and/or prevention according to any of the preceding preferred numbered embodiments, wherein the dosage form additionally comprises least one non-dairy component.

Preferred numbered embodiment 72. The method for treatment and/or prevention according to any of the preceding preferred numbered embodiments, wherein the protein of the AWA comprises beta-lactoglobulin (BLG) in an amount of at least 50% w/w, more preferably at least 70% w/w even more preferably at least 80% w/w and more preferably at least 90% w/w.

Preferred numbered embodiment 73. The method for treatment and/or prevention according to any of the preceding preferred numbered embodiments, wherein the AWA is obtainable by, and e.g. obtained by,
a. providing a whey protein solution:
   having a pH in the range of the 6-9,
   having a weight ratio between total protein and the total content of calcium and magnesium of at least 100
   comprising at least 1-10% w/w BLG relative to the weight of the whey protein solution
   comprising at least 30% BLG relative to total protein
b. subjecting the whey protein solution to heat-treatment which involves heating it to a temperature in the range of 68-180 degrees C. for a duration sufficient to denature at least 25% w/w of the BLG to obtain a suspension containing sWPA, and
c. subjecting the suspension or a protein concentrate thereof to processing that leads to agglomeration of the sWPA, preferably freeze-drying followed by particle size reduction, thereby obtaining agglomerates of soluble whey protein aggregates.

Preferred numbered embodiment 74. A non-therapeutic method, for
a. increasing and/or prolonging the secretion of glucagon-like peptide 1 (GLP-1)
b. providing a biphasic secretion profile of GLP-1
c. providing a delayed secretion profile of GLP-1
d. increasing the sensation of satiety
e. decreasing hunger
f. reducing body weight
g. improving glycemic control
h. reducing glucagon secretion
i. enhancing insulin secretion
j. reducing body and/or liver fat
k. ameliorating muscle atrophy, and/or
l. reducing risk of arteriosclerosis, hypertension, dyslipidemia, and/or cardiovascular events
in a human subject, wherein said method comprises administering to the human subject an effective amount of AWA or a dosage form containing an effective amount of AWA.

Preferred numbered embodiment 75. The non-therapeutic method, according to any of the preceding preferred numbered embodiments, wherein the human subject is an adult, and more preferably a senior.

Preferred numbered embodiment 76. The non-therapeutic method, according to any of the preceding preferred numbered embodiments, wherein the human subject is a young adult.

Preferred numbered embodiment 77. The non-therapeutic method, according to any of the preceding preferred numbered embodiments, wherein the human subject is overweight and more preferably obese.

Preferred numbered embodiment 78. The non-therapeutic method, according to any of the preceding preferred numbered embodiments, wherein said AWA or said dosage form is administered by oral administration.

Preferred numbered embodiment 79. The non-therapeutic method, according to any of the preceding preferred numbered embodiments, wherein said AWA or said dosage form is administered in combination with a meal, preferably as a pre-meal, between 5-30 mins prior to a regular meal, preferably between 30-60 minutes prior to a regular meal, more preferably between 60-90 minutes, preferably between 90-120 minutes, preferably between 120-150 minutes, preferably between 150-180 minutes . . .

Preferred numbered embodiment 80. The non-therapeutic method, according to any of the preceding preferred numbered embodiments, wherein the AWA or the dosage form is administered as a pre-meal, such as between 1-180 minutes prior to a regular meal. Preferred numbered embodiment 81. The non-therapeutic method, according to any of the preceding preferred numbered embodiments, wherein the AWA or the dosage form containing an effective amount of AWA is administered in an amount sufficient to provide a dosage of AWA of at least 0.05 g/kg lean body weight per dosage, preferably at least 0.1 g/kg lean body weight per dosage, more preferably at least 0.3 g/kg lean body weight per dosage, even more preferably at least 0.5 g/kg lean body weight per dosage.

Preferred numbered embodiment 82. The non-therapeutic method, according to any of the preceding preferred numbered embodiments, wherein the AWA or the dosage form containing an effective amount of AWA is administered in an amount sufficient to provide a dosage of AWA of at least 0.05 g/kg lean body weight per dosage, preferably at least 0.1 g/kg lean body weight per dosage, more preferably at least 0.3 g/kg lean body weight per dosage, even more preferably at least 0.5 g/kg lean body weight per dosage, wherein said dosage is administered between 1-5 times a day, such as 1, 2, 3, 4 or 5 times a day.

Preferred numbered embodiment 83. The non-therapeutic method, according to any of the preceding preferred numbered embodiments, wherein said AWA or said dosage form is administered in an amount sufficient to provide a dosage of AWA of 0.05-4 g/kg body weight per day, preferably 0.05-2 g/kg body weight per day, preferably 0.1-1 g/kg body weight per day, more preferably 0.2-0.5 g/kg body weight per day.

Preferred numbered embodiment 84. The non-therapeutic method, according to any of the preceding preferred numbered embodiments, wherein the dosage form is selected from the group consisting of a liquid, a powder, a gel, a capsule, and edible bar.

Preferred numbered embodiment 85. The non-therapeutic method, according to any of the preceding preferred numbered embodiments, wherein the dosage form is a liquid, wherein the liquid is a “ready to drink beverage”.

Preferred numbered embodiment 86. The non-therapeutic method, according to any of the preceding preferred numbered embodiments, wherein the dosage form is a powder intended to be dissolved in a liquid prior to ingestion.

Preferred numbered embodiment 87. The non-therapeutic method, according to any of the preceding preferred numbered embodiments, wherein the dosage form comprises AWA in an amount of at least 30% w/w relative to total protein, more preferably at least 50%, even more preferably at least 60% w/w, and more preferably at least 70% w/w relative to total protein.

Preferred numbered embodiment 88. The non-therapeutic method, according to any of the preceding preferred numbered embodiments, wherein the dosage form comprises AWA in an amount of at least 4 g per intended dosage, more preferably at least 6 g per intended dosage, even more preferably at least 8 g per intended dosage, and most preferably at least 10 g per intended dosage.

Preferred numbered embodiment 89. The non-therapeutic method, according to any of the preceding preferred numbered embodiments, wherein the dosage form comprises AWA in an amount of at least 1 g/100 mL, preferably at least 2 g/100 mL, preferably at least 4 g 100 mL, preferably at least 6 g 100 mL, preferably at least 8 g/100 mL, preferably at least 10 g/100 mL, preferably at least 12 g/100 mL, preferably at least 14 g/100 mL, preferably at least 16 g/100 mL, preferably at least 18 g/100 mL, preferably at least 20 g/100 mL, preferably at least 22 g/100 mL, preferably at least 24 g/100 mL, preferably at least 26 g/100 mL, preferably at least 28 g/100 mL, preferably at least 30 g/100 mL.

Preferred numbered embodiment 90. The non-therapeutic method, according to any of the preceding preferred numbered embodiments, wherein the dosage form comprises AWA in an amount of 1-20 g/100 mL, more preferably 2-18 g/100 mL, even more preferably at least 3-14 g 100 mL, and most preferably at 5-10 g 100 mL.

Preferred numbered embodiment 91. The non-therapeutic method, according to any of the preceding preferred numbered embodiments, wherein the dosage form comprises total protein in an amount of at least 1 g/100 mL, preferably at least 2 g/100 mL, preferably at least 4 g 100 mL, preferably at least 6 g 100 mL, preferably at least 8 g/100 mL, preferably at least 10 g/100 mL, preferably at least 12 g/100 mL, preferably at least 14 g/100 mL, preferably at least 16 g/100 mL, preferably at least 18 g/100 mL, preferably at least 20 g/100 mL, preferably at least 22 g/100 mL, preferably at least 24 g/100 mL, preferably at least 26 g/100 mL, preferably at least 28 g/100 mL, preferably at least 30 g 100 mL.

Preferred numbered embodiment 92. The non-therapeutic method, according to any of the preceding preferred numbered embodiments, wherein the dosage form comprises total protein in an amount of 1-25 g/100 mL, more preferably 2-20 g/100 mL, even more preferably at least 3-16 g/100 mL, and most preferably at 4-14 g/100 mL.

Preferred numbered embodiment 93. The non-therapeutic method, according to any of the preceding preferred numbered embodiments, wherein the dosage form comprises AWA in an amount of at least 10 g/100 g, more preferably at least 15 g/100 g, more preferably at least 20 g/100 g, even more preferably at least 30 g/100 g, and most preferably at least 50 g/100 g.

Preferred numbered embodiment 94. The non-therapeutic method, according to any of the preceding preferred numbered embodiments, wherein the dosage form comprises AWA in an amount of at least 50 g/100 g, more preferably at least 60 g/100 g, more preferably at least 70 g/100 g, even more preferably at least 80 g/100 g, and most preferably at least 90 g/100 g.

Preferred numbered embodiment 95. The non-therapeutic method, according to any of the preceding preferred numbered embodiments, wherein the dosage form comprises AWA in an amount of 10-90 g/100 g, more preferably 15-85 g/100 g, even more preferably at least 20-80 g/100 g, and most preferably at 30-75 g/100 g.

Preferred numbered embodiment 96. The non-therapeutic method, according to any of the preceding preferred numbered embodiments, wherein pH of the dosage form is in the range of 5.5-9.0, more preferably 6.0-8.0, even more preferably 6.2-7.7, and most preferably 6.5-7.5.

Preferred numbered embodiment 97. The non-therapeutic method, according to any of the preceding preferred numbered embodiments, wherein pH of the dosage form is acidic, preferably in the range of 2-5.4, more preferably 2.5-5.0, and most preferably 3.0-4.7.

Preferred numbered embodiment 98. The non-therapeutic method, according to any of the preceding preferred numbered embodiments, wherein the dosage form additionally comprises least one non-dairy component.

Preferred numbered embodiment 99. The non-therapeutic method, according to any of the preceding preferred numbered embodiments, wherein the protein of the AWA comprises beta-lactoglobulin (BLG) in an amount of at least 50% w/w, more preferably at least 70% w/w even more preferably at least 80% w/w and more preferably at least 90% w/w.

Preferred numbered embodiment 100. The non-therapeutic method, according to any of the preceding preferred numbered embodiments, wherein the AWA is obtainable by, and e.g. obtained by,
d. providing a whey protein solution:
   having a pH in the range of the 6-9,
   having a weight ratio between total protein and the total content of calcium and magnesium of at least 100
   comprising at least 1-10% w/w BLG relative to the weight of the whey protein solution
   comprising at least 30% BLG relative to total protein
e. subjecting the whey protein solution to heat-treatment which involves heating it to a temperature in the range of 68-180 degrees C. for a duration sufficient to denature at least 25% w/w of the BLG to obtain a suspension containing sWPA, and
f. subjecting the suspension or a protein concentrate thereof to processing that leads to agglomeration of the sWPA, preferably freeze-drying followed by particle size reduction, thereby obtaining agglomerates of soluble whey protein aggregates.

Preferred numbered embodiment 101. Use of an effective amount of AWA or a dosage form containing an effective amount of AWA for
a. increasing and/or prolonging the secretion of glucagon-like peptide 1 (GLP-1)
b. providing a biphasic secretion profile of GLP-1
c. providing a delayed secretion profile of GLP-1
d. increasing the sensation of satiety
e. decreasing hunger
f. reducing body weight
g. improving glycemic control
h. reducing glucagon secretion
i. enhancing insulin secretion
j. reducing body and/or liver fat
k. ameliorating muscle atrophy, and/or
l. reducing risk of arteriosclerosis, hypertension, dyslipidemia, and/or cardiovascular events
in a human subject.

Preferred numbered embodiment 102. The use of an effective amount of AWA or a dosage form containing an effective amount of AWA according to any of the preceding preferred numbered embodiments, wherein the human subject is an adult, and more preferably a senior.

Preferred numbered embodiment 103. The use of an effective amount of AWA or a dosage form containing an effective amount of AWA according to any of the preceding preferred numbered embodiments, wherein the human subject is a young adult.

Preferred numbered embodiment 104. The use of an effective amount of AWA or a dosage form containing an effective amount of AWA according to any of the preceding preferred numbered embodiments, wherein the human subject is overweight and more preferably obese.

Preferred numbered embodiment 105. The use of an effective amount of AWA or a dosage form containing an effective amount of AWA according to any of the preceding preferred numbered embodiments, wherein said AWA or said dosage form is administered to the human subject by oral administration.

Preferred numbered embodiment 106. The use of an effective amount of AWA or a dosage form containing an effective amount of AWA according to any of the preceding preferred numbered embodiments, wherein said AWA or said dosage form is administered to the human subject by in combination with a meal, preferably as a pre-meal, between 5-30 mins prior to a regular meal, preferably between 30-60 minutes prior to a regular meal, more preferably between 60-90 minutes, preferably between 90-120 minutes, preferably between 120-150 minutes, preferably between 150-180 minutes . . .

Preferred numbered embodiment 107. The use of an effective amount of AWA or a dosage form containing an effective amount of AWA according to any of the preceding preferred numbered embodiments, wherein the AWA or the dosage form is administered to the human subject as a pre-meal, such as between 1-180 minutes prior to a regular meal.

Preferred numbered embodiment 108. The use of an effective amount of AWA or a dosage form containing an effective amount of AWA according to any of the preceding preferred numbered embodiments, wherein the AWA or the dosage form containing an effective amount of AWA is administered to the human subject in an amount sufficient to provide a dosage of AWA of at least 0.05 g/kg lean body weight per dosage, preferably at least 0.1 g/kg lean body weight per dosage, more preferably at least 0.3 g/kg lean body weight per dosage, even more preferably at least 0.5 g/kg lean body weight per dosage.

Preferred numbered embodiment 109. The use of an effective amount of AWA or a dosage form containing an effective amount of AWA according to any of the preceding preferred numbered embodiments, wherein the AWA or the dosage form containing an effective amount of AWA is administered to the human subject in an amount sufficient to provide a dosage of AWA of at least 0.05 g/kg lean body weight per dosage, preferably at least 0.1 g/kg lean body weight per dosage, more preferably at least 0.3 g/kg lean body weight per dosage, even more preferably at least 0.5 g/kg lean body weight per dosage, wherein said dosage is administered between 1-5 times a day, such as 1, 2, 3, 4 or 5 times a day.

Preferred numbered embodiment 110. The use of an effective amount of AWA or a dosage form containing an effective amount of AWA according to any of the preceding preferred numbered embodiments, wherein said AWA or said dosage form is administered to the human subject in an amount sufficient to provide a dosage of AWA of 0.05-4 g/kg body weight per day, preferably 0.05-2 g/kg body weight per day, preferably 0.1-1 g/kg body weight per day, more preferably 0.2-0.5 g/kg body weight per day.

Preferred numbered embodiment 111. The use of a dosage form containing an effective amount of AWA according to any of the preceding preferred numbered embodiments, wherein the dosage form is selected from the group consisting of a liquid, a powder, a gel, a capsule, and edible bar.

Preferred numbered embodiment 112. The use of a dosage form containing an effective amount of AWA according to any of the preceding preferred numbered embodiments, wherein the dosage form is a liquid, wherein the liquid is a “ready to drink beverage”.

Preferred numbered embodiment 113. The use of a dosage form containing an effective amount of AWA according to any of the preceding preferred numbered embodiments, wherein the dosage form is a powder intended to be dissolved in a liquid prior to ingestion.

Preferred numbered embodiment 114. The use of a dosage form containing an effective amount of AWA according to any of the preceding preferred numbered embodiments, wherein the dosage form comprises AWA in an amount of at least 30% w/w relative to total protein, more preferably at least 50%, even more preferably at least 60% w/w, and more preferably at least 70% w/w relative to total protein.

Preferred numbered embodiment 115. The use of a dosage form containing an effective amount of AWA according to any of the preceding preferred numbered embodiments, wherein the dosage form comprises AWA in an amount of at least 4 g per intended dosage, more preferably at least 6 g per intended dosage, even more preferably at least 8 g per intended dosage, and most preferably at least 10 g per intended dosage.

Preferred numbered embodiment 116. The use of a dosage form containing an effective amount of AWA according to any of the preceding preferred numbered embodiments, wherein the dosage form comprises AWA in an amount of at least 1 g/100 mL, preferably at least 2 g/100 mL, preferably at least 4 g 100 mL, preferably at least 6 g 100 mL, preferably at least 8 g/100 mL, preferably at least 10 g/100 mL, preferably at least 12 g/100 mL, preferably at least 14 g/100 mL, preferably at least 16 g/100 mL, preferably at least 18 g/100 mL, preferably at least 20 g/100 mL, preferably at least 22 g/100 mL, preferably at least 24 g/100 mL, preferably at least 26 g/100 mL, preferably at least 28 g/100 mL, preferably at least 30 g/100 mL.

Preferred numbered embodiment 117. The use of a dosage form containing an effective amount of AWA according to any of the preceding preferred numbered embodiments, wherein the dosage form comprises AWA in an amount of 1-20 g/100 mL, more preferably 2-18 g/100 mL, even more preferably at least 3-14 g 100 mL, and most preferably at 5-10 g 100 mL.

Preferred numbered embodiment 118. The use of a dosage form containing an effective amount of AWA according to any of the preceding preferred numbered embodiments, wherein the dosage form comprises total protein in an amount of at least 1 g/100 mL, preferably at least 2 g/100 mL, preferably at least 4 g 100 mL, preferably at least 6 g 100 mL, preferably at least 8 g/100 mL, preferably at least 10 g/100 mL, preferably at least 12 g/100 mL, preferably at least 14 g/100 mL, preferably at least 16 g/100 mL, preferably at least 18 g/100 mL, preferably at least 20 g/100 mL, preferably at least 22 g/100 mL, preferably at least 24 g/100 mL, preferably at least 26 g/100 mL, preferably at least 28 g/100 mL, preferably at least 30 g 100 mL.

Preferred numbered embodiment 119. The use of a dosage form containing an effective amount of AWA according to any of the preceding preferred numbered embodiments, wherein the dosage form comprises total protein in an amount of 1-25 g/100 mL, more preferably 2-20 g/100 mL, even more preferably at least 3-16 g/100 mL, and most preferably at 4-14 g/100 mL.

Preferred numbered embodiment 120. The use of a dosage form containing an effective amount of AWA according to any of the preceding preferred numbered embodiments, wherein the dosage form comprises AWA in an amount of at least 10 g/100 g, more preferably at least 15 g/100 g, more preferably at least 20 g/100 g, even more preferably at least 30 g/100 g, and most preferably at least 50 g/100 g.

Preferred numbered embodiment 121. The use of a dosage form containing an effective amount of AWA according to any of the preceding preferred numbered embodiments, wherein the dosage form comprises AWA in an amount of at least 50 g/100 g, more preferably at least 60 g/100 g, more preferably at least 70 g/100 g, even more preferably at least 80 g/100 g, and most preferably at least 90 g/100 g.

Preferred numbered embodiment 122. The use of a dosage form containing an effective amount of AWA according to any of the preceding preferred numbered embodiments, wherein the dosage form comprises AWA in an amount of 10-90 g/100 g, more preferably 15-85 g/100 g, even more preferably at least 20-80 g/100 g, and most preferably at 30-75 g/100 g.

Preferred numbered embodiment 123. The use of a dosage form containing an effective amount of AWA according to any of the preceding preferred numbered embodiments, wherein pH of the dosage form is in the range of 5.5-9.0, more preferably 6.0-8.0, even more preferably 6.2-7.7, and most preferably 6.5-7.5.

Preferred numbered embodiment 124. The use of a dosage form containing an effective amount of AWA according to any of the preceding preferred numbered embodiments, wherein pH of the dosage form is acidic, preferably in the range of 2-5.4, more preferably 2.5-5.0, and most preferably 3.0-4.7.

Preferred numbered embodiment 125. The use of a dosage form containing an effective amount of AWA according to any of the preceding preferred numbered embodiments, wherein the dosage form additionally comprises least one non-dairy component.

Preferred numbered embodiment 126. The use of an effective amount of AWA or a dosage form containing an effective amount of AWA according to any of the preceding preferred numbered embodiments, wherein the protein of the AWA comprises beta-lactoglobulin (BLG) in an amount of at least 50% w/w, more preferably at least 70% w/w even more preferably at least 80% w/w and more preferably at least 90% w/w.

Preferred numbered embodiment 127. The use of an effective amount of AWA or a dosage form containing an effective amount of AWA according to any of the preceding preferred numbered embodiments, wherein the AWA is obtainable by, and e.g. obtained by,
a. providing a whey protein solution:
   having a pH in the range of the 6-9,
   having a weight ratio between total protein and the total content of calcium and magnesium of at least 100
   comprising at least 1-10% w/w BLG relative to the weight of the whey protein solution
   comprising at least 30% BLG relative to total protein
b. subjecting the whey protein solution to heat-treatment which involves heating it to a temperature in the range of 68-180 degrees C. for a duration sufficient to denature at least 25% w/w of the BLG to obtain a suspension containing sWPA, and
c. subjecting the suspension or a protein concentrate thereof to processing that leads to agglomeration of the sWPA, preferably freeze-drying followed by particle size reduction, thereby obtaining agglomerates of soluble whey protein aggregates.

Preferred numbered embodiment 128. Use of AWA or a dosage form containing an effective amount of AWA in the manufacture of a medicament for the treatment and/or prevention of one or more of
a. type II diabetes,
b. pre-diabetes
c. hyperglycemia
d. Nonalcoholic fatty liver disease (NAFLD)
e. Nonalcoholic steatohepatitis (NASH)
f. overweight
g. obesity,
h. obesity related disorders,
i. muscle atrophy
j. cardio vascular disease
k. arteriosclerosis
l. hypertension
m. dyslipidemia

Preferred numbered embodiment 129. The use of a dosage form containing an effective amount of AWA in the manufacture of a medicament according to any of the preceding preferred numbered embodiments, wherein the dosage form is selected from the group consisting of a liquid, a powder, a gel, a capsule, and edible bar.

Preferred numbered embodiment 130. The use of a dosage form containing an effective amount of AWA in the manufacture of a medicament according to any of the preceding preferred numbered embodiments, wherein the dosage form is a liquid, wherein the liquid is a “ready to drink beverage”.

Preferred numbered embodiment 131. The use of a dosage form containing an effective amount of AWA in the manufacture of a medicament according to any of the preceding preferred numbered embodiments, wherein the dosage form is a powder intended to be dissolved in a liquid prior to ingestion.

Preferred numbered embodiment 132. The use of a dosage form containing an effective amount of AWA in the manufacture of a medicament according to any of the preceding preferred numbered embodiments, wherein the dosage form comprises AWA in an amount of at least 30% w/w relative to total protein, more preferably at least 50%, even more preferably at least 60% w/w, and more preferably at least 70% w/w relative to total protein.

Preferred numbered embodiment 133. The use of a dosage form containing an effective amount of AWA in the manufacture of a medicament according to any of the preceding preferred numbered embodiments, wherein the dosage form comprises AWA in an amount of at least 4 g per intended dosage, more preferably at least 6 g per intended dosage, even more preferably at least 8 g per intended dosage, and most preferably at least 10 g per intended dosage.

Preferred numbered embodiment 134. The use of a dosage form containing an effective amount of AWA in the manufacture of a medicament according to any of the preceding preferred numbered embodiments, wherein the dosage form comprises AWA in an amount of at least 1 g/100 mL, preferably at least 2 g/100 mL, preferably at least 4 g 100 mL, preferably at least 6 g 100 mL, preferably at least 8 g/100 mL, preferably at least 10 g/100 mL, preferably at least 12 g/100 mL, preferably at least 14 g/100 mL, preferably at least 16 g/100 mL, preferably at least 18 g/100 mL, preferably at least 20 g/100 mL, preferably at least 22 g/100 mL, preferably at least 24 g/100 mL, preferably at least 26 g/100 mL, preferably at least 28 g/100 mL, preferably at least 30 g/100 mL.

Preferred numbered embodiment 135. The use of a dosage form containing an effective amount of AWA in the manufacture of a medicament according to any of the preceding preferred numbered embodiments, wherein the dosage form comprises AWA in an amount of 1-20 g/100 mL, more preferably 2-18 g/100 mL, even more preferably at least 3-14 g 100 mL, and most preferably at 5-10 g 100 mL.

Preferred numbered embodiment 136. The use of a dosage form containing an effective amount of AWA in the manufacture of a medicament according to any of the preceding preferred numbered embodiments, wherein the dosage form comprises total protein in an amount of at least 1 g/100 mL, preferably at least 2 g/100 mL, preferably at least 4 g 100 mL, preferably at least 6 g 100 mL, preferably at least 8 g/100 mL, preferably at least 10 g/100 mL, preferably at least 12 g/100 mL, preferably at least 14 g/100 mL, preferably at least 16 g/100 mL, preferably at least 18 g/100 mL, preferably at least 20 g/100 mL, preferably at least 22 g/100 mL, preferably at least 24 g/100 mL, preferably at least 26 g/100 mL, preferably at least 28 g/100 mL, preferably at least 30 g 100 mL.

Preferred numbered embodiment 137. The use of a dosage form containing an effective amount of AWA in the manufacture of a medicament according to any of the preceding preferred numbered embodiments, wherein the dosage form comprises total protein in an amount of 1-25 g/100 mL, more preferably 2-20 g/100 mL, even more preferably at least 3-16 g/100 mL, and most preferably at 4-14 g/100 mL.

Preferred numbered embodiment 138. The use of a dosage form containing an effective amount of AWA in the manufacture of a medicament according to any of the preceding preferred numbered embodiments, wherein the dosage form comprises AWA in an amount of at least 10 g/100 g, more preferably at least 15 g/100 g, more preferably at least 20 g/100 g, even more preferably at least 30 g/100 g, and most preferably at least 50 g/100 g.

Preferred numbered embodiment 139. The use of a dosage form containing an effective amount of AWA in the manufacture of a medicament according to any of the preceding preferred numbered embodiments, wherein the dosage form comprises AWA in an amount of at least 50 g/100 g, more preferably at least 60 g/100 g, more preferably at least 70 g/100 g, even more preferably at least 80 g/100 g, and most preferably at least
90 g/100 g.

Preferred numbered embodiment 140. The use of a dosage form containing an effective amount of AWA in the manufacture of a medicament according to any of the preceding preferred numbered embodiments, wherein the dosage form comprises AWA in an amount of 10-90 g/100 g, more preferably 15-85 g/100 g, even more preferably at least 20-80 g/100 g, and most preferably at 30-75 g/100 g.

Preferred numbered embodiment 141. The use of a dosage form containing an effective amount of AWA in the manufacture of a medicament according to any of the preceding preferred numbered embodiments, wherein pH of the dosage form is in the range of 5.5-9.0, more preferably 6.0-8.0, even more preferably 6.2-7.7, and most preferably 6.5-7.5.

Preferred numbered embodiment 142. The use of a dosage form containing an effective amount of AWA in the manufacture of a medicament according to any of the preceding preferred numbered embodiments, wherein pH of the dosage form is acidic, preferably in the range of 2-5.4, more preferably 2.5-5.0, and most preferably 3.0-4.7.

Preferred numbered embodiment 143. The use of a dosage form containing an effective amount of AWA in the manufacture of a medicament according to any of the preceding preferred numbered embodiments, wherein the dosage form additionally comprises least one non-dairy component.

Preferred numbered embodiment 144. The use of AWA or a dosage form containing an effective amount of AWA in the manufacture of a medicament according to any of the preceding preferred numbered embodiments, wherein the protein of the AWA comprises beta-lactoglobulin (BLG) in an amount of at least 50% w/w, more preferably at least 70% w/w even more preferably at least 80% w/w and more preferably at least 90% w/w.

Preferred numbered embodiment 145. The use of AWA or a dosage form containing an effective amount of AWA in the manufacture of a medicament according to any of the preceding preferred numbered embodiments, wherein the AWA is obtainable by, and e.g. obtained by,
a. providing a whey protein solution:
having a pH in the range of the 6-9,
having a weight ratio between total protein and the total content of calcium and magnesium of at least 100
comprising at least 1-10% w/w BLG relative to the weight of the whey protein solution
comprising at least 30% BLG relative to total protein
b. subjecting the whey protein solution to heat-treatment which involves heating it to a temperature in the range of 68-180 degrees C. for a duration sufficient to denature at least 25% w/w of the BLG to obtain a suspension containing sWPA, and
c. subjecting the suspension or a protein concentrate thereof to processing that leads to agglomeration of the sWPA, preferably freeze-drying followed by particle size reduction, thereby obtaining agglomerates of soluble whey protein aggregates.

### EXAMPLES

### Example 1: Agglomerates of Soluble Whey Protein Aggregates

### 1.1 AWA Prepared by Freeze-Drying

Agglomerates of soluble whey protein aggregates (AWA) were prepared by freeze-drying of a suspension of soluble whey protein aggregates which was prepared from a whey protein isolate powder (SPI) obtained from native milk serum protein. The chemical composition of the SPI is summarized in Table 1: Composition of the whey protein isolate (SPI) obtained from native milk serum protein used for preparing agglomerates of soluble whey protein aggregates.

| Components | % w/w |
|---|---|
| Ash | 2.13 |
| Fat | 0.23 |
| Lactose | 0.14 |
| pH | 6.69 |
| Protein | 91.7 |
| alpha lactalbumin of | 25.3 |
| total protein | |
| beta-lactoglobulin of | 53.3 |
| total protein | |
| other of total protein | 1.97 |
| Total solids | 95.6 |
| Protein of total solids | 95.9 |
| Calcium | 0.403 |
| Mg | 0.062 |
| Na | 0.368 |
| K | 0.014 |
| P | 0.16 |
| Cl | ≤0.04^{*)}. |

The suspension of soluble whey protein aggregates was prepared by the following procedure:
37.8 kg of demineralized water was transferred to a Scanima Mixer,
The demineralized water was heated to 45-50 degrees C.,
2.2 kg SPI powder prepared form milk serum was added to the demineralized water and was dissolve by slow agitation for 1 hours,
The pH of the solution was adjusted to pH 7.4 by slowly adding 6% NaOH in small dosages,
The pH adjusted solution was then heated to 80 degrees C. and held at this temperature for 20 minutes to produce soluble whey protein aggregates,
After the 20 minutes holding time the temperature was reduced to approx. 6 degrees by indirect cooling,
The cooled suspension was placed in plastic trays that were stored at −20 to −25 degrees C.,
The frozen suspension was then freeze dried for 24 hours under vacuum (approx. 1 mBar, starting at −40 degrees C. and gradually added heated) which caused sublimation of frozen water to steam and resulted in a dried product,
The dried product was finally ground to form a powder containing agglomerates of soluble whey protein aggregates.

The content of agglomerated soluble whey protein aggregates present after the freeze drying process was estimated to 80% w/w relative to total protein.

TABLE 1
Composition of the whey protein isolate (SPI) obtained
from native milk serum protein used for preparing
agglomerates of soluble whey protein aggregates.

| Components | % w/w |
|---|---|
| Ash | 2.13 |
| Fat | 0.23 |
| Lactose | 0.14 |
| pH | 6.69 |
| Protein | 91.7 |
| alpha lactalbumin of | 25.3 |
| total protein | |
| beta-lactoglobulin of | 53.3 |
| total protein | |
| other of total protein | 1.97 |
| Total solids | 95.6 |
| Protein of total solids | 95.9 |
| Calcium | 0.403 |
| Mg | 0.062 |
| Na | 0.368 |
| K | 0.014 |
| P | 0.16 |
| Cl | ≤0.04^{*)} |

^{*)}Below the detection limit

Gelling properties of the agglomerates (AWA-f) prepared in Ex 1.1 were compared to soluble whey protein aggregates (sWPA) by Glucono-delta-lactone (GDL) acidification.

The samples for acidification were prepared by the following procedure: 20 g AWA-f powder was mixed with demineralized water to total of 500 g to get a 4% protein. The solution was set to hydrate overnight in the fridge.

A 4% sWPA solution was prepared by diluting the 5% sWPA (prepared the same day) in demineralized water.

The acidification and simultaneous gel strength measurement was carried out as follow: 200 g of the sample was set to equilibrate at 42° C. in water bath. 5 g GDL was added to get 2.5% GDL while stirring and recording pH: First the solution was stirred for 1 min to ensure mixing into the sample. Then 19.6 mL was transferred to rheometer (Anton Paar with CC27 set up) and measurement was started. Rheometer settings: 1 Hz, 0.05%, 1 min per point for 1 hr at 42° C. (first two points not measured)

The rest of the sample was incubated in water bath (without stirring) and pH measured every 1 min with pH logger. After measurement the pH and gel strength (G′, storage modulus) were correlated.

The sWPA-sample showed a rapid increase in gel strength in the beginning of acidification but subsequently dissolution of the gel at lower pH as shown by a decrease in gel strength.

The AWA-f sample showed a delayed increase in gel strength ending with a high gel strength at lower pH values ultimately exceeding the gel strength of sWPA at pH less than about 4.

### 1.2 Calcium-Induced AWA Formation

Six samples of calcium-induced AWA (AWA-C) were prepared from a suspension of soluble whey protein aggregates (sWPA) which was prepared from a whey protein isolate powder (SPI) obtained from native milk serum protein, as disclosed in ex 1.1.

The 6 samples were prepared to have a molar Calcium: protein ratio of 2.5:1, 3:1, 3.5:1, 4:1, 4.5:1, and 5:1 and conversely the samples were referred to as AWA-C2.5. AWA-C3, AWA-C3.5 AWA-C4, AWA-C4.5 and AWA-C5, respectively.

More specifically, the AWA-C samples were prepared by taking 40 mL samples of the sWPA suspension and equilibrating them at 75° C. in a water bath. Then 10 mL CaCl₂) solution was added (each sWPA sample was mixed with a calcium solution giving the final mixture the desired molar ratio between calcium and protein) while stirring to end up with a final protein concentration of 4% w/w. The mixtures were incubated at the 75° C. for 3 min with stirring. Subsequently, mixtures cooled to 23 degrees C. and were allowed to incubate for approximately 3 hours at this temperature to provide samples AWA-C2.5 to AWA-C5, which were analyzed immediately.

Samples AWA-C2.5 to AWA-C5 were all liquid compositions with a slight development of opaqueness and AWA-C3.5 to AWA-C5 furthermore contained visible agglomerates.

A larger sample of AWA-C5 (AWA-C5b) was further prepared in the same manner by equilibrating 160 mL sWPA suspension at 75° C. and adding 40 mL CaCl₂) solution under stirring to get at final molar stoichiometry of 5:1 Calcium: protein.

The gelling properties and pH stability of the agglomerates of AWA-C5b prepared were compared to the sWPA suspension and as well as to an AWA-f suspension prepared from Ex 1.1, by Glucono-delta-lactone (GDL) acidification.

The samples were treated with two different GDL concentration (1% and 5% w/w) to be able to analyze the samples at different end-pH's after 1 hr incubation at 37° C. The acidification was done as follow: 3 samples of 40 g were taken from each of the sWPA suspension, AWA-f, and AWA-C5b and were equilibrated at 37° C. (i.e. 9 samples in total). One of the three samples of each protein sample type left untreated, and the other two samples mixed with 0.4 g GDL (approx. 1% w/w) and 2 g GDL (approx. 5% w/w) respectively. The samples were stirred for 1 min to distribute the GDL evenly. The samples were then incubated at 37° C. for 1 hr without stirring. Subsequently, 20 g from each sample were centrifuged at 3000 g for 5 minutes at room temperature, after which the total protein content of the supernatant was determined according to Analysis 7 of WO2021136785A1. Additionally, the final pH was measured and was found to be:
pH 7.1±0.3 for the samples where no GDL had been added
pH 4.2±0.1 for the samples where 1% w/w GDL had been added, and
pH 2.9±0.1 for the samples where 5% w/w GDL had been added.

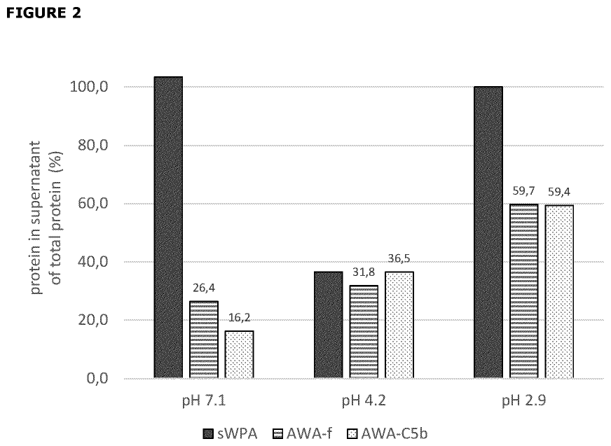

The results of the Example 1.2 have been plotted in FIG. 2. As can be seen from the figure the sWPA sample had all protein in the supernatant after centrifugation at pH 7.1, but when it was acidified to pH 4.2 it had formed a gel and most of the protein was in the pellet. However, when the pH reached 2.9 all gel had dissolved and the protein was found in the supernatant again. This behaviour was expected from gel strength measurement described in example 1.1.

FIG. 2 further shows that for both AWA-f and AWA-C5b the protein concentration was low in the supernatant at pH 7.1, since the AWA had sedimented during the centrifugation. During the acidification only a small amount of the protein was released to the supernatant. At pH 4.2 approx. 35% of the protein of AWA-f and AWA C5b was in the supernatant which means that the remaining 65% was in the centrifugation pellet (and therefore in agglomerate form). Even at pH 2.9, where all protein of the sWPA-sample had returned to the supernatant, the AWA-f and the AWA-C5b sample still had approx. 40% in the form agglomerate form (and approx. only 60% in the supernatant). This demonstrates that the AWA prepared by both freeze-drying (AWA-f) and by Ca-induced sWPA agglomeration (AWA-C5b) have a better structural stability and are capable of providing and maintaining a higher gel strength under the acidic conditions that are found in the human stomach than comparable amounts of sWPA.

A high gel strength of the contents of the stomach has previously been linked to an increased sensation of fullness and satiety and it is therefore evident that effective amounts of AWA can be used to increase the sensation of satiety and therefore is useful for treatment and prevention of e.g. obesity and diseases and disorders related to obesity. GLP-1 boosting effect of AWA described in Example 2 further contributes to its medical usefulness.

### Example 2: Physiological Response to AWA Intake in Human Subjects

### 2.1 Protein Ingredients

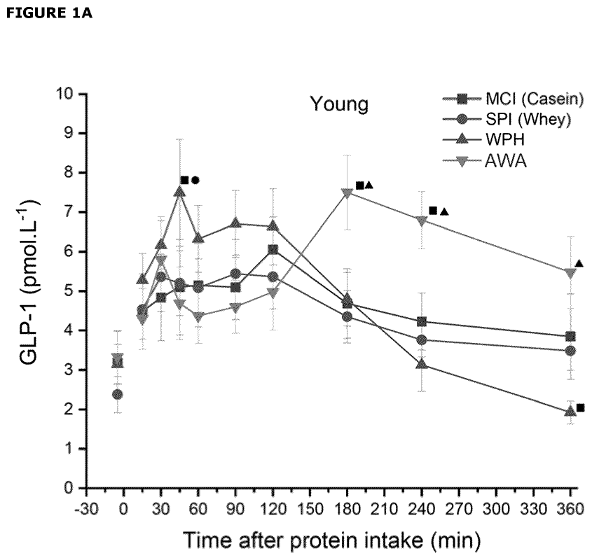

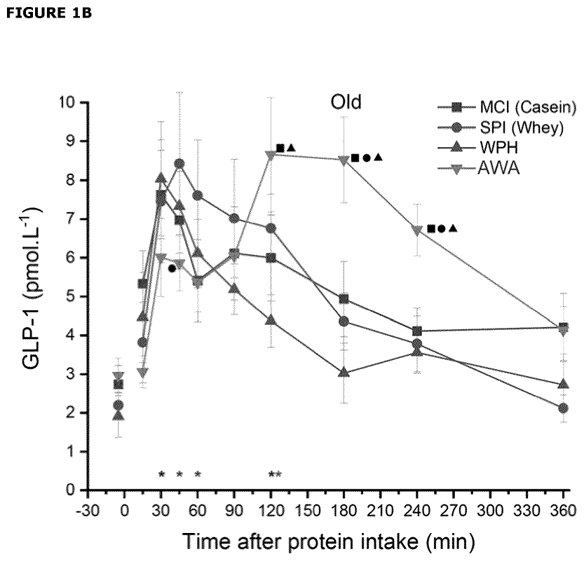

The following protein ingredient (in powder form) were subjected to in vivo testing:
the ground, AWA-containing product obtained from Example 1.1 (referred to as AWA in FIGS. 1A and 1B) was compared to
the original SPI with the composition shown in Table 1 (referred to as SPI in FIGS. 1A and 1B;)
   a protein hydrolysate of the SPI prepared by enzymatic digestion to a degree of hydrolysis of approx. 23 (referred to as WPH in FIGS. 1A and 1B)
a micellar casein isolate containing 86.5% total protein, 95% total casein of total protein, 5% whey protein of total protein, 1.5% fat; 0.4% lactose, 8.2% ash (referred to as MCI in FIGS. 1A and 1B)

### 2.2 Subject Characteristics

Ten healthy volunteers, 5 females and 5 males, were included in both the young (Young) and the elderly group (Old). Exclusion criteria were chronical illness, smoking, alcohol abuse, lactose intolerance, irregular menstrual cycle and intake of drugs known to affect protein metabolism. Before the clinical trials started the subjects underwent a DEXA scan to determine body composition. Age, weight, height, BMI and fat free mass were 22.7±0.8 and 76.3±1.5 years; 72±4 and 74±5 kg; 23.3±0.9 and 25.8±0.9 kg/m2; 171±2 and 169±4 cm; 50±3 and 47±3 kg for the Young and Old, respectively. Age range in Old: 70.4-82.4 years, Young: 18.2-25.0 years.

TABLE 2
Subjects in study (mean ± SEM)

| | Female | Young | male | Female | Old | male |
|---|---|---|---|---|---|---|
| Age (years) | 23.6 ± 0.6 | 22.7 ± 0.8 | 21.8 ± 1.4 | 77.2 ± 2.0 | 76.3 ± 1.5 | 74.2 ± 2.1 |
| Weight (kg) | 65 ± 4 | 72 ± 4 | 79 ± 4 | 64 ± 3 | 74 ± 5 | 88 ± 5 |
| BMI | 22.2 ± 1.5 | 23.3 ± 0.9 | 24.5 ± 0.9 | 24.0 ± 0.8 | 25.8 ± 0.9 | 28.1 ± 1.0 |
| Height | 171 ± 2 | 175 ± 2 | 180 ± 3 | 163 ± 2 | 169 ± 4 | 177 ± 5 |
| FFM | 42 ± 1 | 50 ± 3 | 57 ± 4 | 40 ± 2 | 47 ± 3 | 58 ± 1 |

### 2.3 Experimental Day

The subjects were instructed to refrain from strenuous physical activities two days prior to the experimental day. On the experimental day itself the subjects arrived overnight fasted and were only allowed to drink water ad libitum during the experimental day. At their arrival they were placed in a hospital bed and intravenous catheters were placed in the antecubital veins; one in each arm for blood sampling and tracer infusion, respectively. A background blood sample was taken before starting a primed continuous infusion of different stable-isotope labeled amino acid tracers purchased from Cambridge Isotope Laboratories (Andover, USA). Blood samples were obtained 100 and 115 min after the start of the tracer infusions. After the 115 min baseline sample was obtained the in water dissolved/dispersed protein ingredients (0.6 g/kg Fat Free Mass) were provided and subjects encourage to ingest the liquid solution in about 5 min after which the beaker glass was rinsed with 100 ml water to removed protein connected to the wall and the subject asked to ingest this as well. Blood samples were taken 15, 30, 45, 60, 90,120, 180, 210, 240, 270, 300 and 360 min after the subjects had started the protein drink intake. The blood samples were collected into chilled EDTA tubes and centrifuged for 10 min. The plasma was transferred to Eppendorf tubes and stored at −80° C. until analysis.

### 2.4 Analyses

Plasma samples were analyzed at the Clinical Metabolomics Core Facility, (Rigshospitalet, Denmark to determine the concentration and enrichment of circulating amino acids according to Børno et al (8). Hormones were determined by ELISA (Mercodia, Uppsala, Sweden), Total glucagon-lile-peptide-1 (GLP-1) were measured with luminescence.

### 2.5 Statistical Analysis

The data are expressed as means±SEM. A Two-Way Repeated Measures ANOVA with time and protein as factors was used to compare the effect of the milk protein ingredients kinetics over time. When a significant interaction between the factors was determined, the Tukey test was used to find the time points were the effect of the ingredients were significantly different. The statistical analysis was performed in OriginPro 2020. The statistical significance level was set to P<0.05.

### 2.6 Results

After ingestion of AWA, the GLP-1 plasma response was biphasic and exhibited a first rapid small peak at 30 min, followed by a delayed/prolonged larger increase/peak. Notably, in the young subjects, plasma GLP-1 was higher than the other protein sources from 180-360 min. In the old subjects, this significantly higher GLP-1 after AWA was apparent after 120 min.

For the specific effect of the AWA, comparison to the native form (SPI) is the most relevant.

In young subjects, the peak GLP-1 concentration (i.e. Cmax) after the AWA meal was roughly the same as after administering WPH, but much higher than any peak for MCI and SPI. In old subjects the peak GLP-1 for AWA was roughly the same as any of WPH, MCI or SPI. Meanwhile, the overall GLP-1 response (area under the curve) was increased and prolonged in both young and old subjects compared to any of WPH, MCI and SPI.

As documented in Example 1.2, AWA prepared by e.g. Ca-induced sWPA-agglomeration has the same type structural stability as the AWA prepared by prepared by freeze-drying (e.g. according to Example 1.1). It is therefore evident that the ability of AWA to increase and prolong the GLP-1 response in humans upon oral administration is independent of how the AWA has been produced.

### REFERENCES

Abdulla, H., Phillips, B. E., Wilkinson, D. J., Limb, M., Jandova, T., Bass, J. J., Rankin, D., Cegielski, J., Sayda, M., Crossland, H., Williams, J. P., Smith, K., Idris, I., & Atherton, P. J. (2020). Glucagon-like peptide 1 infusions overcome anabolic resistance to feeding in older human muscle. Aging Cell, 19 (9), 1-12. doi.org/10.1111/acel.13202
Bornø A, van Hall G. Quantitative amino acid profiling and stable isotopically labeled amino acid tracer enrichment used for in vivo human systemic and tissue kinetics measurements. J Chromatogr B Analyt Technol Biomed Life Sci. 2014; 951-952:69-77.
Cruz-Jentoft et al 2019. Sarcopemia: revised European consensus on definition and daignosis. Age and Ageing 2019; 48:16-31. doi: 10.1093/ageing/afyl69
Flint, A., Andersen, G., Hockings, P., Johansson, L., Morsing, A., Sundby Palle, M., Vogl, T., Loomba, R., & Plum-Morschel, L. (2021). Randomised clinical trial: semaglutide versus placebo reduced liver steatosis but not liver stiffness in subjects with non-alcoholic fatty liver disease assessed by magnetic resonance imaging. Alimentary Pharmacology and Therapeutics, 54 (9), 1150-1161. doi.org/10.1111/apt.16608
Gurjar, A. A., Kushwaha, S., Chattopadhyay, S., Das, N., Pal, S., China, S. P., Kumar, H., Trivedi, A. K., Guha, R., Chattopadhyay, N., & Sanyal, S. (2020). Long acting GLP-1 analog liraglutide ameliorates skeletal muscle atrophy in rodents. Metabolism: Clinical and Experimental, 103, 154044. doi.org/10.1016/j.metabol.2019.154044
Hong, Y., Lee, J. H., Jeong, K. W., Choi, C. S., & Jun, H. S. (2019). Amelioration of muscle wasting by glucagon-like peptide-1 receptor agonist in muscle atrophy. Journal of Cachexia, Sarcopenia and Muscle, 10 (4), 903-918. doi.org/10.1002/jcsm.12434
Kaneto, H., Kimura, T., Shimoda, M., Obata, A., Sanada, J., Fushimi, Y., Nakanishi, S., Mune, T., & Kaku, K. (2021). Favorable effects of GLP-1 receptor agonist against pancreatic B-cell glucose toxicity and the development of arteriosclerosis: “the earlier, the better” in therapy with incretin-based medicine. International Journal of Molecular Sciences, 22 (15), 1-11. doi.org/10.3390/ijms22157917
Krieger, J. P. (2020). Intestinal glucagon-like peptide-1 effects on food intake: Physiological relevance and emerging mechanisms. Peptides, 131 (June), 170342. doi.org/10.1016/j.peptides.2020.170342
Love, K. M., Liu, J., Regensteiner, J. G., Reusch, J. E. B., & Liu, Z. (2020). GLP-1 and insulin regulation of skeletal and cardiac muscle microvascular perfusion in type 2 diabetes. Journal of Diabetes, 12 (7), 488-498. doi.org/10.1111/1753-0407.13045
Nguyen, T. T. N., Choi, H., & Jun, H. S. (2020). Preventive Effects of Dulaglutide on Disuse Muscle Atrophy Through Inhibition of Inflammation and Apoptosis by Induction of Hsp72 Frontiers Expression. in Pharmacology, 11 (February), 1-11. doi.org/10.3389/fphar.2020.00090
Patel Chavez, C., Cusi, K., & Kadiyala, S. (2022). The Emerging Role of Glucagon-like Peptide-1 Receptor Agonists for the Management of NAFLD. The Journal of Clinical Endocrinology and Metabolism, 107 (1), 29-38. doi.org/10.1210/clinem/dgab578
Patel, v. J., Joharapurkar, A. A., Shah, G. B. & Jain, M. R. (2014) Effect of GLP-1 based therapies on diabetic dyslipidemia. Curr Diabetes Rev 10 (4): 238-50. doi: 10.2174/1573399810666140707092506.
Subaran, S. C., Sauder, M. A., Chai, W., Jahn, L. A., Fowler, D. E., Aylor, K. W., Basu, A., & Liu, Z. (2014). GLP-1 at physiological concentrations recruits skeletal and cardiac muscle microvasculature in healthy humans. Clinical Science, 127 (3), 163-170. doi.org/10.1042/CS20130708
Zhao, Y., Vanhoutte, P. M., & Leung, S. W. S. (2015). Vascular nitric oxide: Beyond eNOS. Journal of 83-94. Pharmacological Sciences, 129 (2), doi.org/10.1016/j.jphs.2015.09.002

1. Agglomerates of soluble whey protein aggregates (AWA) or a dosage form containing an effective amount of AWA for use as a medicament.

2. The AWA or the dosage form containing an effective amount of AWA according to claim 1, for use in treating and/or preventing one or more disorders linked to GLP-1 blood levels in a human subject.

3. The AWA or the dosage form containing an effective amount of AWA, for use in treatment and/or prevention according to claim 2, wherein said AWA facilitates to one or more of
i. increased sensation of satiety and/or decreasing hunger
ii. reduced body weight
iii. improved glycemic control
iv. reduced glucagon secretion
v. enhanced insulin secretion
vi. reduced body and/or liver fat
vii. ameliorated muscle atrophy
viii. reduced risk of arteriosclerosis, hypertension, dyslipidemia, and/or cardiovascular events
in the human subject.

4. The AWA or the dosage form containing an effective amount of AWA, for use in treatment and/or prevention according to claim 2 or 3, wherein said disorders linked to GLP-1 blood levels are selected from one or more of:
a. overweight
b. obesity,
c. obesity related disorders,
d. type II diabetes,
e. pre-diabetes
f. hyperglycemia
g. Nonalcoholic fatty liver disease (NAFLD)
h. Nonalcoholic steatohepatitis (NASH)
i. muscle atrophy
j. cardio vascular disease
k. arteriosclerosis
l. hypertension
m. dyslipidemia

5. The AWA or the dosage form containing an effective amount of AWA, for use in treatment and/or prevention according to claim 2, wherein said one or more disorders are selected from one or more of overweight, obesity, and obesity related disorders.

6. The AWA or the dosage form containing an effective amount of AWA, for use in treatment and/or prevention according to claim 5, wherein said AWA facilitates increased sensation of satiety and/or decreased hunger in the human subject.

7. The AWA or the dosage form containing an effective amount of AWA, for use in treatment and/or prevention according to claim 2, wherein said one or more disorders are selected from one or more of type II diabetes, pre-diabetes, and hyperglycemia.

8. The AWA or the dosage form containing an effective amount of AWA, for use in treatment and/or prevention according to claim 7, wherein said AWA facilitates improved glycemic control, reduced glucagon secretion, and/or enhanced insulin secretion in the human subject.

9. The AWA or the dosage form containing an effective amount of AWA, for use in treatment and/or prevention according to claim 2, wherein said one or more disorders are selected from one or more of nonalcoholic fatty liver disease (NAFLD) and nonalcoholic steatohepatitis (NASH).

10. The AWA or the dosage form containing an effective amount of AWA, for use in treatment and/or prevention according to claim 9, wherein said AWA facilitates reduced body and/or liver fat in the human subject.

11. The AWA or the dosage form containing an effective amount of AWA, for use in treatment and/or prevention according to claim 2, wherein said one or more disorders is muscle atrophy.

12. The AWA or the dosage form containing an effective amount of AWA, for use in treatment and/or prevention according to claim 11, wherein said AWA facilitates increased muscle microvascular perfusion.

13. The AWA or the dosage form containing an effective amount of AWA, for use in treatment and/or prevention according to claim 2, wherein said one or more disorders are selected from one or more of cardio vascular disease, arteriosclerosis, hypertension, and dyslipidemia.

14. The AWA or the dosage form containing an effective amount of AWA, for use in treatment and/or prevention according to any one of claims 1-13, wherein the AWA or the dosage form containing an effective amount of AWA is for oral administration to the human subject.

15. The AWA or the dosage form containing an effective amount of AWA, for use in treatment and/or prevention according to any one of claims 1-14, wherein the AWA or the dosage form containing an effective amount of AWA is administered as a pre-meal, such as between 1-180 minutes prior to a regular meal.

16. The AWA or the dosage form containing an effective amount of AWA, for use in treatment and/or prevention according to any one of claims 1-15, wherein the AWA or the dosage form containing an effective amount of AWA is administered in an amount sufficient to provide a dosage of AWA of at least 0.05 g/kg lean body weight per dosage.

17. The AWA or the dosage form containing an effective amount of AWA, for use in treatment and/or prevention according to claim 16, wherein said dosage is administered between 1-5 times a day.

18. The dosage form containing an effective amount of AWA, for use according to any one of claims 1-17, wherein the dosage form is selected from the group consisting of a liquid, a powder, a gel, a capsule, and an edible bar.

19. The dosage form containing an effective amount of AWA, for use according to any one of claims 1-18, comprising AWA in an amount of at least 30% w/w relative to total protein.

20. The dosage form containing an effective amount of AWA, for use according to any one of claims 1-19, comprising AWA in an amount of at least 4 g per intended dosage.

21. A dosage form containing an effective amount of AWA, and preferably comprising AWA in an amount of at least 30% w/w relative to total protein.

22. The dosage form according to claim 21 wherein the dosage form is selected from the group consisting of a liquid, a powder, a gel, a capsule, and an edible bar.

23. The dosage form according to claim 21 or 22 having a pH in the range of 5.5-9.0, more preferably 6.0-8.0, even more preferably 6.2-7.7, and most preferably 6.5-7.5.

24. The dosage form according to claim 21 or 22 having a pH in the range of 2-5.4, more preferably 2.5-5.0, and most preferably 3.0-4.7.

25. The dosage form according to any one of claims 21-24 comprising AWA in an amount at least 50% relative to total protein, even more preferably at least 60% w/w, and more preferably at least 70% w/w relative to total protein.

26. The dosage form according to any one of claims 21-25 comprising AWA in an amount of at least 4 g per intended dosage, more preferably at least 6 g per intended dosage, even more preferably at least 8 g per intended dosage, and most preferably at least 10 g per intended dosage.

27. The dosage form according to any one of claims 21-26 which is a liquid dosage form, preferably a read-to-drink liquid.

28. The dosage form according to claim 27 which is a pasteurized or sterilized liquid.

29. The dosage form according to claim 27 or 28 wherein the liquid dosage form comprises AWA in an amount of 1-20 g/100 mL, more preferably 2-18 g/100 mL, even more preferably at least 3-14 g 100 mL, and most preferably at 5-10 g 100 mL.

30. The dosage form according to any one of claims 27-29 wherein the liquid dosage form comprises total protein in an amount of 1-25 g/100 mL, more preferably 2-20 g/100 mL, even more preferably at least 3-16 g/100 mL, and most preferably at 4-14 g/100 mL.

31. The dosage form according to any one of claims 21-26 which is a powder, preferably a powder intended to be dissolved in a liquid prior to ingestion.

32. The dosage form according to claim 31 wherein the powder dosage form comprises AWA in an amount of at least 10 g/100 g, more preferably at least 15 g/100 g, more preferably at least 20 g/100 g, even more preferably at least 30 g/100 g, and most preferably at least 50 g/100 g.

33. The dosage form according to claim 31 or 32 wherein the powder dosage form comprises AWA in an amount of at least 50 g/100 g, more preferably at least 60 g/100 g, more preferably at least 70 g/100 g, even more preferably at least 80 g/100 g, and most preferably at least 90 g/100 g.

34. The dosage form according to any one of claims 31-33 wherein the powder dosage form comprises AWA in an amount of 10-90 g/100 g, more preferably 15-85 g/100 g, even more preferably at least 20-80 g/100 g, and most preferably at 30-75 g/100 g.

35. The dosage form according to any one of claims 31-34 wherein the powder dosage form is a nutritional powder comprising:
AWA in an amount of at least 30% relative to total protein, more preferably at least 50%, even more preferably at least 60% w/w, and more preferably at least 70% w/w relative to total protein, and
at least one non-dairy component.

36. The dosage form according to any one of claims 31-35 wherein the powder dosage form is a nutritional powder comprising AWA in an amount of at least
30% w/w relative to total protein and at least one non-dairy protein.

37. The dosage form according to any one of claims 21-36 wherein the preparation of the dosage form involves mixing an AWA-containing composition obtainable by the method according to one or more of claims 41-61.

38. The dosage form according to any one of claims 21-37 wherein the AWA used to prepare the dosage form is provided by an AWA-containing composition obtainable by the method according to one or more of claims 41-61.

39. The AWA or the dosage form containing an effective amount of AWA for use according to any one of claims 1-20, or the dosage form according to any one of claims 21-36, wherein the AWA is obtainable by, and e.g. obtained by,
a. providing a whey protein solution:
having a pH in the range of the 6-9,
having a weight ratio between total protein and the total content of calcium and magnesium of at least 100
comprising at least 1-10% w/w BLG relative to the weight of the whey protein solution
comprising at least 30% BLG relative to total protein
b. subjecting the whey protein solution to heat-treatment which involves heating it to a temperature in the range of 68-180 degrees C. for a duration sufficient to denature at least 25% w/w of the BLG to obtain a suspension containing sWPA, and
c. subjecting the suspension or a protein concentrate thereof to processing that leads to agglomeration of the sWPA, thereby obtaining agglomerates of soluble whey protein aggregates.

40. The AWA or the dosage form containing an effective amount of AWA, for use according to claim 39, wherein the processing in step c is freeze-drying followed by particle size reduction of the freeze-dried product, thereby obtaining agglomerates of soluble whey protein aggregates.

41. A method of producing a composition comprising AWA, comprising the steps of
a. providing a whey protein solution:
having a pH in the range of the 6-9,
having a weight ratio between total protein and the total content of calcium and magnesium of at least 100
comprising at least 1-10% w/w BLG relative to the weight of the whey protein solution
comprising at least 30% BLG relative to total protein
b. subjecting the whey protein solution to heat-treatment which involves heating it to a temperature in the range of 68-180 degrees C. for a duration sufficient to denature at least 25% w/w of the BLG to obtain a suspension containing sWPA, and
c. subjecting the suspension or a protein concentrate thereof to processing that leads to agglomeration of the sWPA, thereby obtaining a composition comprising agglomerates of soluble whey protein aggregates.

42. The method according to claim 41, wherein the processing of step c. involves one or more of:
freeze-drying,
increasing the levels of divalent metal cations and/or increased levels of monovalent metal cations,
lowering the pH,
concentration, preferably by one or more of reverse osmosis, nanofiltration, ultrafiltration, microfiltration, evaporation, and a combination thereof.

43. The method according to claim 41 or 42 wherein the processing of step c. that leads to agglomeration of the sWPA involves freeze-drying.

44. Method according to any one of claims 41-43 wherein the agglomeration step is followed by particle size reduction of the freeze-dried product.

45. The method according to any one of claims 41-44 wherein the processing of step c. that leads to agglomeration of the sWPA involves addition of divalent metal ions, preferably calcium and/or magnesium.

46. The method according to any one of claims 41-45 wherein the added divalent metal ions are provided by water-soluble salts of calcium and/or magnesium, preferably CaCl₂), MgCl₂, CaSO₄, MgSO₄, CaCO₃, MgCO₃, calcium phosphate and/or magnesium phosphate.

47. The method according to any one of claims 41-46 wherein the divalent metal ions are added in an amount sufficient to obtain a molar ratio between the total amount of divalent metal ion and the amount of total protein of at least 1:1, more preferably at least 2:1, even more preferably at least 3:1, and most preferably at least 4:1.

48. The method according to any one of claims 41-47 wherein the divalent metal ions are added in an amount sufficient to obtain a molar ratio between the total amount of divalent metal ion and the amount of total protein of 1:1-100:1, more preferably 2:1-60:1, even more preferably 3:1-40:1, and more preferably 3:1-30:1.

49. The method according to any one of claims 41-48 wherein the divalent metal ions are added in an amount sufficient to obtain a molar ratio between the total amount of divalent metal ion and the amount of total protein of 1:1-20:1, more preferably 2:1-16:1, even more preferably 3:1-12:1, and more preferably 3:1-10:1.

50. The method according to any one of claims 41-49 wherein the liquid enriched with divalent metal ions has a molar percentage of calcium ions relative to the total molar content of divalent metal ions of at least 50% more preferably at least 70% even more preferably at least 80%, and most preferably at least 90%.

51. The method according to any one of claims 41-50 wherein the liquid enriched with divalent metal ions has a temperature during the incubation in the range of 10-100 degrees C., more preferably 20-90 degrees C., even more preferably 35-85 degrees C., and most preferably 40-80 degrees C.

52. The method according to any one of claims 41-50 wherein the liquid enriched with divalent metal ions has a temperature during the incubation in the range of 10-90 degrees C., more preferably 15-85 degrees C., even more preferably 20-85 degrees C., and most preferably 20-80 degrees C.

53. The method according to any one of claims 41-52 wherein the liquid enriched with divalent metal ions is incubated for at least 1 minute, more preferably at least 2 minutes, and most preferably for at least 3 minutes.

54. The method according to any one of claims 41-53 wherein the liquid enriched with divalent metal ions is incubated for at least 0.5 hour, more preferably at least 1 hour, and most preferably for at least 2 hours.

55. The method according to any one of claims 41-54 wherein the liquid enriched with divalent metal ions is incubated for 1 minute to 48 hours, more preferably 2 minutes to 36 hours, and most preferably 3 minutes to 24 hours.

56. The method according to any one of claims 41-55 wherein the liquid enriched with divalent metal ions is incubated for 0.5 hour to 48 hours, more preferably 1 hour to 36 hours, and most preferably 2 hours to 24 hours.

57. The method according to any one of claims 41-56 wherein liquid enriched with divalent metal ions is agitated, e.g. by stirring, during the incubation.

58. The method according to any one of claims 41-57 wherein step c. furthermore involves subjecting the incubated liquid enriched with divalent metal ions to one or more of:
a concentration step, preferably one or more of evaporation, reverse osmosis, nanofiltration, ultrafiltration and/or microfiltration, and
a drying step, preferably involving spray-drying.

59. The method according to claim 58 wherein the drying step involve preheating the liquid to be dried to a temperature in the range of 50-80 degrees C. prior to the actual spraying which converts the liquid to be dried to droplets from which water evaporates.

60. The method according to claim 58 or 59 wherein the liquid to be dried has a weight percentage of AWA relative to the weight of the liquid of 2-10% w/w, more preferably 3-9% w/w, and most preferably 4-8% w/W.

61. The method according to any one of claims 58-60 wherein the liquid to be dried has a weight percentage of AWA relative to total protein of at least 40% w/W, more preferably at least 50% w/w, even more preferably at least 60% w/w, and most preferably at least 70% w/w.
